(19) United States
(12) Patent Application Publication
(10) Pub. No.: US 2003/0161849 A1
(43) Pub. Date: Aug. 28, 2003

(54) Cosmetic and dermatological preparations in the form of O/W emulsions, comprising an amino-substituted hydroxybenzophenone

(76) Inventors: Thomas Heidenfelder, Dannstadt (DE); Thomas Wunsch, Speyer (DE); Valerie Andre, Ludwigshafen (DE)

Correspondence Address: Herbert B. Keil, KEIL & WEINKAUF, 1350 Connecticut Ave., N.W, Washington, DC, 20036, US

(21) Appl. No.: 10/234,158

(22) Filed: Sep. 5, 2002

(30) Foreign Application Priority Data
Sep. 7, 2001 (DE) 10143962.8

(51) Int. Cl. A61K 31/24; A61K 7/00

(52) U.S. Cl. 424/401; 514/541

(57) ABSTRACT

Cosmetic or dermatological preparations in the form of an O/W emulsion, comprising an amino-substituted hydroxybenzophenone of the formula I.

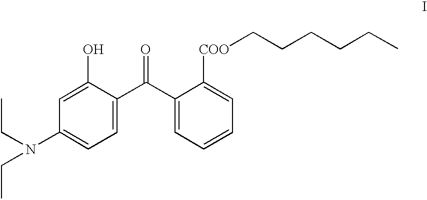

The present invention relates to cosmetic and dermatological preparations in the form of O/W emulsions, comprising an amino-substituted hydroxybenzophenone. In one advantageous embodiment, the present invention relates to a use which allows the stability of O/W emulsions to be increased.

The skin is the largest human organ. Amongst its many functions (for example for temperature regulation and as a sensory organ) the barrier function, which prevents the skin (and ultimately the entire organism) from drying out, is doubtless the most important. At the same time, the skin acts as a protective device against the penetration and absorption of external substances. This barrier function is effected by the epidermis, which, as the outermost layer, forms the actual protective sheath against the environment. Being about one tenth of the total thickness, it is also the thinnest layer of the skin.

The epidermis is a stratified tissue in which the outer layer, the horny layer (Stratum corneum), is the part which is of significance for the barrier function. The Elias skin model, which is currently recognized in the specialist field (P. M. Elias, Structure and Function of the Stratum Corneum Permeability Barrier, Drug Dev. Res. 13, 1988, 97-105), describes the horny layer as a two-component system, similar to a brick wall (bricks and mortar model). In this model, the horny cells (corneocytes) correspond to the bricks, and the lipid membrane in the intercellular spaces, which is of complex composition, corresponds to the mortar. This system is essentially a physical barrier to hydrophilic substances, but, because of its narrow and multilayered structure, can equally, however, also only be passed by lipophilic substances with difficulty.

The present invention relates, in a particular embodiment, to cosmetic or pharmaceutical preparations having a reduced feeling of stickiness, to processes for their preparation, and also to the use of active ingredients for reducing the feeling of stickiness of cosmetic preparations.

Apart from its barrier action against external chemical and physical influences, the epidermal lipids also contribute to the holding together of the horny layer and have an effect on the smoothness of the skin. In contrast to the sebaceous gland lipids, which do not form a continuous film on the skin, the epidermal lipids are distributed over the entire horny layer.

The extremely complex interaction of the moisture-binding substances and of the lipids of the upper layers of the skin is very important for the regulation of skin moisture. For this reason, cosmetics generally comprise, in addition to balanced lipid mixtures and water, water-binding substances.

In addition to the chemical composition, however, the physical behavior of these substances is also of importance. The development of very biocompatible emulsifiers and surfactants is therefore desirable. Products formulated therewith aid the liquid-crystalline organization of the intercellular lipids of the Stratum corneum, thereby improving the barrier properties of the horny layer. It is particularly advantageous if their molecular constituents consist of substances which are naturally occurring in the epidermis.

Cosmetic skin care primarily means that the natural function of the skin as a barrier against environmental influences (e.g. dirt, chemicals, microorganisms) and against the loss of endogenous substances (e.g. water, natural fats, electrolytes) is strengthened or rebuilt.

If this function is impaired, increased resorption of toxic or allergenic substances or attack by microorganisms may result, leading to toxic or allergic skin reactions.

Another aim of skin care is to compensate for the loss by the skin of lipids and water caused by daily washing. This is particularly important when the natural regeneration ability is insufficient. Furthermore, skincare products should protect against environmental influences, in particular against sun and wind, and delay skin aging.

Medicinal topical compositions generally comprise one or more medicaments in an effective concentration. For the sake of simplicity, in order to distinguish clearly between cosmetic and medicinal use and corresponding products, reference is made to the legal provisions of the Federal Republic of Germany (e.g. Cosmetics Directive, Foods and Drugs Act).

Customary cosmetic forms of application are emulsions. This term generally means a heterogeneous system of two liquids which are immiscible or miscible only to a limited extent with one another, which are usually referred to as phases. One is in the form of droplets (disperse or internal phase), while the other liquid forms a continuous (coherent or internal) phase. Less common forms of application are multiple emulsions, i.e. those which, in the droplets of the dispersed (or discontinuous) phase, comprise for their part droplets of a further dispersed phase, e.g. W/O/W emulsions and O/W/O emulsions.

If the oil phase is finely distributed in the water phase, then this is an oil-in-water emulsion (O/W emulsion, e.g. milk). The basic character of an O/W emulsion is determined by the water, i.e. it is generally less greasy on the skin, is rather matting and absorbs more rapidly into the skin than a W/O emulsion.

The person skilled in the art is naturally familiar with a large number of options of formulating stable O/W preparations for cosmetic or dermatological use, for example in the form of creams and ointments, which are spreadable in the range from room temperature to skin temperature, or as lotions and milks, which are more flowable in this temperature range.

The stability of emulsions is dependent inter alia on their viscosity, in particular on the viscosity of the external phase. An emulsion becomes unstable when the finely dispersed particles collect together again to form relatively large aggregates, and the droplets which are in contact coalesce. This process is referred to as coalescence. The more viscous the external phase of the emulsion, the slower the process of coalescence.

Accordingly, O/W emulsions are usually stabilized by thickeners, which increase the viscosity of the aqueous phase. Examples which are suitable for this purpose are polyacrylates (carbomer) and further organic thickeners. A disadvantage of this method of improving stability is the sensitivity of these formulations toward electrolytes. In addition, predominantly higher-viscosity formulations (such as creams or ointments) can of course be prepared in this manner.

Emulsions of “liquid” (=flowable) consistency are used in cosmetics, for example as care lotion, cleansing lotion, face lotion or hand lotion. They generally have a viscosity of from about 2000 mPa·s to about 10,000 mPa·s. The stability of flowable emulsions is deserving of particular attention since the considerably greater mobility of the particles promotes more rapid coalescence.

Even liquid emulsions of the prior art—since they too generally comprise thickeners—are not stable toward relatively high electrolyte concentrations, which manifests itself in phase separation. It is, however, frequently desirable to use certain electrolytes, such as, for example, water-soluble UV filters, in order to be able to utilize the other physical, chemical or physiological properties thereof. Although in many cases appropriate choice of the emulsifier system can provide remedies to a certain extent, other disadvantages then arise just as often.

The discussed disadvantages can, for example, lie in the fact that emulsifiers, like ultimately any chemical substance, can in individual cases trigger allergic reactions or reactions based on oversensitivity of the user, although the use of customary cosmetic emulsifiers is of course generally entirely without risk.

In order to be able to ensure the metastability of emulsions, interface-active substances, i.e. emulsifiers, are usually necessary. The use per se of customary cosmetic emulsifiers is entirely acceptable. Nevertheless, emulsifiers, as ultimately any chemical substance, may in certain cases cause allergic reactions or reactions based on oversensitivity of the user. For example, it is known that in some particularly sensitive people, certain light dermatoses are triggered by certain emulsifiers and simultaneous action of sunlight.

It is possible to prepare emulsifier-free preparations which, for example, have, in an aqueous phase, dispersed oil droplets, similar to an O/W emulsion. A prerequisite for this may be that the continuous aqueous phase has a gel framework which stabilizes the dispersed phase, and other conditions besides. Such systems are sometimes called hydrodispersions or oleodispersions depending on which is the disperse phase and which is the continuous phase.

For cosmetic technology, it is, however, neither necessary nor possible to dispense with emulsifiers altogether, especially since there is a certain choice of particularly mild emulsifiers. However, the prior art lacks a satisfactorily broad range of such emulsifiers which would then also significantly broaden the, application spectrum of correspondingly mild cosmetic preparations which are tolerated by the skin.

The harmful effect of the ultraviolet part of solar radiation on the skin is generally known. While rays having a wavelength of less than 290 nm (the UVC region) are absorbed by the ozone layer in the earth's atmosphere, rays in the range between 290 nm and 320 nm, the UVB region, cause erythema, simple sunburn or even burns of varying severity.

The erythema activity maximum of sunlight is given as the relatively narrow region around 308 nm.

Numerous compounds are known for protecting against UVB radiation; these are derivatives of 3-benzylidenecamphor, of 4-aminobenzoic acid, of cinnamic acid, of salicylic acid, of benzophenone and also of 2-phenyl-benzimidazole.

It is also important to have available filter substances for the range between about 320 nm and about 400 nm, the UVA region, since its rays can also cause damage. For a long time it has been incorrectly assumed that the long-wave UV-A radiation having a wavelength of between 320 nm and 400 nm has only a negligible biological action and that, accordingly, the UV-B rays are responsible for most photodamage to the human skin. In the meantime, however, it has been proven by numerous studies that UV-A radiation is much more harmful than UV-B radiation with regard to the triggering of photodynamic, specifically phototoxic, reactions and chronic changes in the skin. Also, the harmful effect of UV-B radiation can be further intensified by UV-A radiation.

Thus, it has inter alia been shown that UV-A radiation on its own under very normal everyday conditions is enough to damage collagen and elastin fibers, which are of essential importance for the structure and strength of the skin, within a short period. This leads to chronic light-induced changes in the skin—the skin “ages” prematurely. The clinical appearance of skin aged by light includes, for example, wrinkles and lines, and an irregular, furrowed relief. In addition, the parts affected by light-induced skin aging can have irregular pigmentation. Even the formation of brown patches, keratoses and even carcinomas or malignant melanomas is possible. Skin aged prematurely as a result of everyday UV exposure is also characterized by lower activity of the Langerhans cells and slight, chronic inflammation.

Approximately 90% of the ultraviolet radiation which reaches the earth consists of UV-A rays. While the UV-B radiation varies widely depending on numerous factors (e.g. time of year and day or degree of latitude), the UV-A radiation remains relatively constant day after day irrespective of the time of year and day or geographical factors. Additionally, the majority of UV-A radiation penetrates the living epidermis, while about 70% of the UV-B rays are retained by the horny layer.

Preventive protection against UV-A rays, for example by applying light protection filter substances in the form of a cosmetic or dermatological formulation to the skin, is therefore of fundamental importance.

Generally speaking, the light absorption behavior of light protection filter substances is very well known and documented, not least because most industrialized countries have positive lists for the use of such substances, which impose very strict standards on the documentation. For the concentration of the substances in the finished formulations, the absorbance values can at best be a guide, since interaction with substances within the skin or the surface of the skin itself may result in imponderables. In addition, it is usually difficult to estimate beforehand how uniformly and thickly the filter substance is distributed in and on the horny layer of the skin.

To test the UV-A protection performance, use is usually made of the IPD method (IPD≡immediate pigment darkening). Similarly to the determination of the sun protection factor, this method gives a value which indicates how much longer the skin protected with the light protection composition can be irradiated with UV-A radiation before the pigmentation which occurs is the same as for unprotected skin.

Another test method which has become established throughout Europe is the Australian standard AS/NZS 2604:1997. Here, the absorption of the preparation in the UV-A region is measured. In order to satisfy the standard, the preparation must absorb at least 90% of the UV-A radiation in the region 320-360 nm.

The use concentration of known light protection filter substances which, in particular, also exhibit high filter action in the UV-A region is often limited by the very fact that they are combined with other substances which are in the form of solids. There are therefore certain formulation difficulties in achieving relatively high sun protection factors and UV-A protection performance.

Since light protection filter substances are generally expensive and since some light protection filter substances are additionally difficult to incorporate into cosmetic or dermatological preparations in relatively high concentrations, an object of the invention was to obtain, in a simple and cost-effective manner, preparations which, despite having unusually low concentrations of conventional UV-A light protection filter substances, nevertheless achieve acceptable or even high UV-A protection performance.

UV radiation can, however, also lead to photochemical reactions, where the photochemical reaction products interfere with the skin's metabolism. Such photochemical reaction products are predominantly free-radical compounds, for example hydroxyl radicals. Undefined free-radical photoproducts which form in the skin itself can also exhibit uncontrolled secondary reactions as a result of their high reactivity. However, singlet oxygen, a non-free-radical excited state of the oxygen molecule, can also arise during UV irradiation, as can short-lived epoxides and many others. Singlet oxygen, for example, differs from normal triplet oxygen (free-radical ground state) by virtue of its increased reactivity. However, excited, reactive “free-radical” triplet states of the oxygen molecule also exist.

In order to prevent these reactions, antioxidants and/or free-radical scavengers can be additionally incorporated into the cosmetic or dermatological formulations.

The compounds which are used as light protection agents for cosmetic and dermatological light protection formulations are mostly characterized per se by good light protection. However, they have the disadvantage that it is sometimes difficult to incorporate them into such formulations in a satisfactory manner.

The sun protection factor (SPF) indicates how much longer the skin protected with the light protection composition can be irradiated before the erythema reaction which occurs is the same as for unprotected skin (i.e. ten times as long comPared with unprotected skin for an SPF=10).

In any case, the consumer expects, on the one hand, reliable information from the manufacturer regarding the sun protection factor, not least because of the discussion about the “hole in the ozone layer” which has become a topic of public interest, and on the other hand there is a tendency by the consumer toward relatively high and high sun protection factors.

Since light protection filter substances are generally expensive and since some light protection filter substances are additionally difficult to incorporate into cosmetic or dermatological preparations in relatively high concentrations, an object of the invention was to obtain, in a simple and cost-effective manner, preparations which, despite having unusually low concentrations of conventional light protection filter substances, nevertheless achieve acceptable or even high SPF values.

It was also an object of the present invention to conceive cosmetic or dermatological light protection preparations which are characterized by increased care action.

It was therefore surprising and could not have been foreseen by the person skilled in the art that cosmetic or dermatological preparations in the form of an O/W emulsion, comprising an amino-substituted hydroxybenzophenone of the formula I, overcome the disadvantages of the prior art.

It was surprising that preparations within the meaning of the present invention would lead to unexpectedly high light protection factors and UV-A protection.

It could not have been foreseen by the person skilled in the art either that the preparations according to the invention

would be more effective moisturizing preparations,

would better promote skin smoothing,

would be characterized by better care action,

would have higher stability toward the crystallization of the raw materials used,

would be characterized by better biocompatibility,

would be characterized by a better feel on the skin and by greater cosmetic elegance,

would be characterized over a broad cosmetic variability and would be able to be formulated over broad consistency and viscosity ranges from 400 mPas to >20,000 mPas

than the preparations of the prior art.

The preparations according to the invention have very good cosmetic properties, in particular with regard to stickiness, and have very good skin comPatibility and skin care performance.

The preparations according to the invention comprise an amino-substituted hydroxybenzophenone of the formula I.

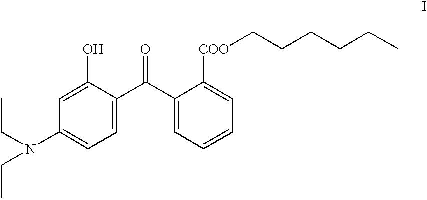

This class of substance is described, inter alia, in DE-A-199 17 906. Compound I is characterized in particular by its good solubility in oil, its good processability into cosmetic preparations and by a good feel on the skin.

The amount of compound of the formula I used in the finished cosmetic or dermatological preparations is advantageously chosen from the range 0.01% by weight to 20% by weight, preferably from 0.1 to 10% by weight, particularly preferably from 1 to 7% by weight, based on the total weight of the preparations.

Basic constituents of the preparations according to the invention which may be used are:

water or aqueous solutions

aqeuous ethanolic solutions

natural oils and/or chemically modified natural oils and/or synthetic oils;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used.

For the purposes of the present disclosure, the expression “lipids” is sometimes used as a generic term for fats, oils, waxes and the like, said expression being entirely commonplace to the person skilled in the art. The terms “oil phase” and “lipid phase” are also used synonymously.

Oils and fats differ from one another in their polarity, which is difficult to define. It has already been proposed to adopt the interfacial tension toward water as a measure of the polarity index of an oil or of an oily phase. Then, the lower the interfacial tension between this oily phase and water, the greater the polarity of the oily phase in question. According to the invention, the interfacial tension is regarded as one possible measure of the polarity of a given oil component.

The interfacial tension is the force which acts on an imaginary line one meter in length in the interface between two phases. The physical unit for this interfacial tension is conventionally calculated from the force/length relationship and is usually expressed in mN/m (millinewtons divided by meters). It has a positive sign if it endeavours to reduce the interface. In the converse case, it has a negative sign. For the purposes of the present invention, lipids are regarded as polar if their interfacial tension toward water is less than 30 mN/m.

Polar oils are for example those from the group of lecithins and of fatty acid triglycerides, namely the triglycerol esters of saturated and/or unsaturated, branched and/or unbranched alkanecarboxylic acids having a chain length of from 8 to 24, in particular 12 to 18, carbon atoms. The fatty acid triglycerides can, for example, advantageously be chosen from the group of synthetic, semisynthetic and natural oils, such as, for example, olive oil, sunflower oil, soya oil, groundnut oil, rapeseed oil, almond oil, palm oil, coconut oil, castor oil, wheatgerm oil, grapeseed oil, thistle oil, evening primrose oil, macadamia nut oil and the like, provided the condition required in the main claim is observed.

Other polar oil components can be chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, and from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, such as, for example, jojoba oil.

In addition, the oily phase can be advantageously chosen from the group of dialkyl ethers, the group of saturated or unsaturated, branched or unbranched alcohols. It is particularly advantageous if the oily phase of the preparations according to the invention contains C₁₂-₁₅-alkyl benzoate or consists entirely of the latter.

In addition, the oil phase can advantageously be chosen from the group of Guerbet alcohols. Guerbet alcohols are named after Marcel Guerbet who described their preparation for the first time. They are formed according to the equation

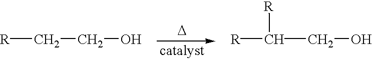

by oxidation of an alcohol to an aldehyde, by aldol condensation of the aldehyde, elimination of water from the aldol and hydrogenation of the allyl aldehyde. Guerbet alcohols are liquid even at low temperatures and effect virtually no skin irritations. They can be used advantageously as fatting, superfatting and also refatting constituents in skincare and haircare compositions.

The use of Guerbet alcohols in cosmetics is known per se. Such species are then in most cases characterized by the structure

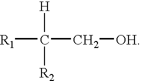

Here, R₁ and R₂ are usually unbranched alkyl radicals.

According to the invention, the Guerbet alcohol(s) is/are advantageously chosen from the group in which

R₁ is propyl, butyl, pentyl, hexyl, heptyl or octyl and

R₂ is hexyl, heptyl, octyl, nonyl, decyl, undecyl, dodecyl, tridecyl or tetradecyl.

Guerbet alcohols preferred according to the invention are 2-butyloctanol with the following chemical structure

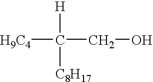

which is available, for example, under the tradename Isofol® 12 from Condea Chemie GmbH, and 2-hexyldecanol with the following chemical structure

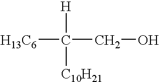

which is available, for example, under the tradename Isofol® 16 from Condea Chemie GmbH.

Mixtures of Guerbet alcohols according to the invention can also be used advantageously according to the invention. Mixtures of 2-butyloctanol and 2-hexyldecanol are available, for example, under the tradename Isofol® 14 from Condea Chemie GmbH.

The total amount of Guerbet alcohols in the finished cosmetic or dermatological preparations is advantageously chosen from the range up to 25.0% by weight, preferably 0.5 to 15.0% by weight, based on the total weight of the preparations.

Any mixtures of such oil and wax components can also be used advantageously for the purposes of the present invention. It may also be advantageous to use waxes, for example cetyl palmitate, as the sole lipid component of the oil phase.

Nonpolar oils are, for example, those which are chosen from the group of branched and unbranched hydrocarbons and hydrocarbon waxes, in particular Vaseline (petrolatum), paraffin oil, squalane and squalene, polyolefins and hydrogenated polyisobutenes. Of the polyolefins, polydecenes are the preferred substances.

Fatty and/or wax components which are to be used advantageously according to the invention can be chosen from the group of vegetable waxes, animal waxes, mineral waxes and petrochemical waxes. Examples which are favorable according to the invention are candelilla wax, carnauba wax, Japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microcrystalline waxes provided the conditions required in the main claim are observed.

Other advantageous fatty and/or wax components are chemically modified waxes and synthetic waxes, such as, for example, those obtainable under the trade names Syncrowax® HRC (glyceryl-tribehenate) and Syncrowax® AW 1C (C₁₈-C₃₆ fatty acid) from CRODA GmbH, and montan ester waxes, Sasol waxes, hydrogenated jojoba waxes, synthetic or modified beeswaxes (e.g. dimethicone copolyol beeswax and/or C₃₀₋₅₀ alkyl beeswax), polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, trihydroxystearin, fatty acids, fatty acid esters, and glycol esters, such as, for example, C₂₀-C₄₀-alkyl stearate, C₂₀-C₄₀-alkylhydroxystearoyl stearate and/or glycol montanate. Also advantageous are certain organosilicon compounds, which have similar physical properties to the specified fatty and/or wax components, such as, for example, stearoxytrimethylsilane provided the conditions required in the main claim are observed.

According to the invention, the fatty and/or wax components can be present either individually or as a mixture.

Any desired mixtures of such oil and wax components can also be used advantageously for the purposes of the present invention.

The oily phase is advantageously chosen from the group consisting of 2-ethylhexyl isostearate, octyldodecanol, isotridecyl isononanoate, isoeicosane, 2-ethylhexyl cocoate, C₁₂-C₁₅-alkyl benzoate, caprylic/capric triglyceride, dicaprylyl ether provided the conditions required in the main claim are observed.

Particularly advantageous mixtures are those of octyldodecanol, caprylic/capric triglyceride, dicaprylyl ether or mixtures of C₁₂-C₁₅-alkyl benzoate and 2-ethylhexyl isostearate, those of C₁₂-C₁₅-alkyl benzoate and isotridecyl isononanoate, and those of C₁₂-C₁₅-alkyl benzoate, 2-ethylhexyl isostearate and isotridecyl isononanoate, provided the conditions required in the main claim are observed.

Table 1 below lists lipids which are advantageous according to the invention as individual substances and also as mixtures with one another. The corresponding interfacial tensions toward water are given in the last column. It is, however, also advantageous to use mixtures of greater or lesser polar components and the like.

| Trade name | INCI name | (m/Nm) |
|---|---|---|
| Isofol ® 14 T | Butyl Decanol + Hexyl Decanol + Hexyl | 27.6 |
| | Octanol + Butyl Octanol | |
| Isofol ® 16 | Hexyl Decanol | 24.3 |
| Eutanol ® G | Octyldodecanol | 24.8 |
| Cetiol ® OE | Dicaprylyl Ether | 22.1 |
| Miglyol ® 812 | Caprylic/Capric Triglyceride | 21.3 |
| Cegesoft ® C24 | Octyl Palmitate | 23.1 |
| Isopropyl | Isopropyl Stearate | 21.9 |
| stearate | | |
| Estol ® 1540 EHC | Octyl Octanoate | 30.0 |
| Finsolv ® TN | C₁₂—C₁₅ Alkyl Benzoate | 21.8 |
| Cetiol ® SN | Cetearyl Isonoanoate | 28.6 |
| Dermofeel ® BGC | Butylene Glycol Dicaprylate/Dicapate | 21.5 |
| Trivent ® OCG | Tricaprylin | 20.2 |
| MOD | Octyldodeceyl Myristate | 22.1 |
| Cosmacol ® ETI | Di-C₁₂—C₁₃ Alkyl Tartrate | 29.4 |
| Miglyol ® 829 | Caprylic/Capric Diglyceryl Succinate | 29.5 |
| Prisorine ® 2036 | Octyl Isostearate | 29.7 |
| Tegosoft ® SH | Stearyl Heptanoate | 28.7 |
| Abil ® Wax 9840 | Cetyl Dimethicone | 25.1 |
| Cetiol ® LC | Coco-Caprylate/Caprate | 24.8 |
| IPP | Isopropyl Palmitate | 22.5 |
| Luvitol ® EHO | Cetearyl Octanoate | 28.6 |
| Cetiol ® 868 | Octyl Stearate | 28.4 |

It may likewise be advantageous to choose some or all of the oil phase of the preparations according to the invention from the group of cyclic and/or linear silicones which are also referred to for the purposes of the present disclosure as “silicone oils”. Such silicones or silicone oils may be present as monomers which are generally characterized by structural elements as follows:

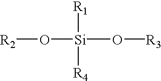

Silicones having two or more siloxyl units which are to be used advantageously according to the invention are generally characterized by structural elements as follows:

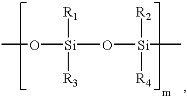

where the silicon atoms may be substituted by identical or different alkyl radicals and/or aryl radicals, which are represented in general terms by the radicals R₁ to R₄, where the number of different radicals is not necessarily limited to 4. m may assume values from 2 to 200,000.

Cyclic silicones to be used advantageously according to the invention are generally characterized by the structural elements as follows

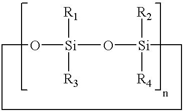

where the silicon atoms may be substituted by identical or different alkyl radicals and/or aryl radicals, which are represented here in general terms by the radicals R₁ to R₄, where the number of different radicals is not necessarily limited to 4. n can assume values of 3/2 to 20. Fractional values for n take into consideration that uneven numbers of siloxyl groups may be present in the cycle.

Phenyltrimethicone is advantageously chosen as silicone oil. Other silicone oils, for example dimethicone, phenyldimethicone, cyclomethicone (octamethylcyclotetrasiloxane), for example hexamethylcyclotrisiloxane, polydimethylsiloxane, poly(methylphenylsiloxane), cetyldimethicone, behenoxydimethicone can also be used advantageously for the purposes of the present invention.

Mixtures of cyclomethicone and isotridecyl isononanoate, and those of cyclomethicone and 2-ethylhexyl isostearate are also advantageous.

It is, however, also advantageous to choose silicone oils of similar constitution, such as the compounds referred to above whose organic side chains have been derivatized, for example polyethoxylated and/or polypropoxylated. These include, for example, polysiloxane-polyalkyl-polyether copolymers such as cetyldimethicone copolyol, (cetyldimethicone copolyol (and) polyglyceryl-4 isostearate (and) hexyl laurate).

O/W emulsions according to the invention can advantageously be prepared using customary O/W emulsifiers, if desired with the aid of W/O emulsifiers or other coemulsifiers.

If desired, O/W emulsions corresponding to the present invention further comprise one or more emulsifiers, if desired advantageously chosen from the group of the following substances which generally act as W/O emulsifiers:

lecithin, lanolin, microcrystalline wax (Cera microcristallina) in a mixture with paraffin oil (Paraffinum liquidum), ozokerite, hydrogenated castor oil, polyglyceryl-3 oleate, wool wax acid mixtures, wool wax alcohol mixtures, pentaerythrithyl isostearate, polyglyceryl-3 diisostearate, beeswax (Cera alba) and stearic acid, sodium dihydroxycetylphosphate in a mixture with isopropyl hydroxycetyl ether, methylglucose dioleate, methylglucose dioleate in a mixture with hydroxystearate and beeswax, mineral oil in a mixture with petrolatum and ozokerite and glyceryl oleate and lanolin alcohol, petrolatum in a mixture with ozokerite and hydrogenated castor oil and glyceryl isostearate and polyglyceyl-3 oleate, PEG-7 hydrogenated castor oil, ozokerite and hydrogenated castor oil, polyglyceryl-4 isostearate, polyglyceryl-4 isostearate in a mixture with cetyldimethicone copolyol and hexyl laurate, laurylmethicone copolyol, cetyldimethicone copolyol, acrylate/C₁₀-C₃₀-alkyl acrylate crosspolymer, Poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoyl polyglyceryl-3 diisostearate, polyglyceryl-2 dipolyhydroxystearate, polyglyceryl-3 dipolyhydroxystearate, polyglyceryl-4 dipolyhydroxystearate, polyglyceryl-3 dioleate.

If desired, O/W emulsions corresponding to the present invention comprise one or more coemulsifiers, particularly advantageously chosen from the group of the following substances which generally act as O/W emulsifiers:

Glyceryl stearate in a mixture with ceteareth-20, ceteareth-25, ceteareth-6 in a mixture with stearyl alcohol, cetylstearyl alcohol in a mixture with PEG-40 castor oil and sodium cetylstearyl sulfate, triceteareth-4 phosphate, sodium cetylstearyl sulfate, lecithin trilaureth-4 phosphate, laureth-4 phosphate, stearic acid, propylene glycol stearate SE, PEG-25 hydrogenated castor oil, PEG-54 hydrogenated castor oil, PEG-6 caprylic/capric glycerides, glyceryl oleate in a mixture with propylene glycol, ceteth-2, ceteth-20, polysorbate 60, glyceryl stearate in a mixture with PEG-100 stearate, laureth-4, ceteareth-3, isostearyl glyceryl ether, cetylstearyl alcohol in a mixture with sodium cetylstearyl sulfate, laureth-23, steareth-2, glyceryl stearate in a mixture with PEG-30 stearate, PEG-40 stearate, glycol distearate, PEG-22 dodecyl glycol copolymer, polyglyceryl-2 PEG-4 stearate, ceteareth-20, methylglucose sesquistearate, steareth-10, PEG-20 stearate, steareth-2 in a mixture with PEG-8 distearate, steareth-21, steareth-20, isosteareth-20, PEG-45/dodecyl glycol copolymer, methoxy-PEG-22/dodecyl glycol copolymer, PEG-20 glyceryl stearate, PEG-8 beeswax, polyglyceryl-2 laurate, isostearyl diglyceryl succinate, stearamidopropyl PG dimonium chloride phosphate, glyceryl stearate SE, ceteth-20, triethyl citrate, PEG-20 methylglucose sesquistearate, ceteareth-12, glyceryl stearate citrate, cetyl phosphate, triceteareth-4 phosphate, trilaureth-4 phosphate, polyglyceryl methylglucose distearate, potassium cetyl phosphate, isosteareth-10, polyglyceryl-2 sesquiisostearate, ceteth-10, oleth-20, isoceteth-20, glyceryl stearate in a mixture with ceteareth-20, ceteareth-12, cetylstearyl alcohol and cetyl palmitate, cetylstearyl alcohol in a mixture with PEG-20 stearate, PEG-30 stearate, PEG-40 stearate, PEG-100 stearate.

It may also be advantageous for the purposes of the present invention, particularly when the oil phase of the preparations consists at least partially of silicone oils, to use silicone emulsifiers. The silicone emulsifiers may advantageously be chosen from the group of interface-active substances from the group of alkylmethicone copolyols and/or alkyl dimethicone copolyols, particularly from the group of compounds characterized by the following chemical structure:

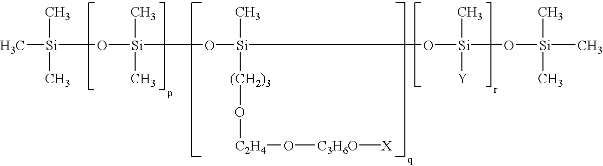

in which X and Y, independently of one another, are chosen from the group H and the branched and unbranched alkyl groups, acyl groups and alkoxy groups having 1 to 24 carbon atoms, p is a number from 0 to 200, q is a number from 1 to 40, and r is a number from 1 to 100.

An example of silicone emulsifiers which are to be used particularly advantageously for the purposes of the present invention are dimethicone copolyols, which are sold by Th. Goldschmidt AG under the tradenames ABIL® B 8842, ABIL® B 8843, ABIL® B 8847, ABIL® B 8851, ABIL® B 8852, ABIL® B 8863, ABIL® B 8873 and ABIL® B 88183.

Another example of interface-active substances to be used particularly advantageously for the purposes of the present invention is cetyldimethicone copolyol, which is sold by Th. Goldschmidt AG under the tradename ABIL® EM 90.

Another example of interface-active substances to be used particularly advantageously for the purposes of the present invention is the cyclomethiconedimethicone copolyol, which is sold by Th. Goldschmidt AG under the tradename ABIL® EM 97.

In addition, an emulsifier which has proven especially advantageous is laurylmethicone copolyol, which is available under the tradename Dow Corning® 5200 Formulation Aid from Dow Corning Ltd.

The total amount of emulsifiers used according to the invention in the cosmetic or dermatological preparations according to the invention is advantageously chosen from the range from 0.1 to 10.0% by weight, preferably 0.5 to 5.0% by weight, based on the total weight of the preparations.

For the purposes of the present invention, sprayable O/W emulsions, in particular O/W microemulsions, are particularly advantageous.

The droplet diameters of the customary “simple”, i.e. nonmultiple, emulsions are in the range from about 1 μm to about 50 μm. Such “macroemulsions” are, without further coloring additives, milky-white in color and opaque. Finer “macroemulsions” whose droplet diameters are in the range from about 0.5 μm to about 1 μm are, again without coloring additives, bluish-white in color and opaque. Such “macroemulsions” usually have a high viscosity.

Only micellar and molecular solutions having particle diameters of less than about 10⁻² μm, but which are no longer to be regarded as true emulsions, have a clear and transparent appearance.

The droplet diameter of microemulsions for the purposes of the present invention, by contrast, is in the range from about 50 to about 500 nm. Such microemulsions are bluish-white in color to translucent and mostly of low viscosity. The viscosity of many microemulsions of the O/W type is comParable with that of water.

An advantage of microemulsions is that, in the disperse phase, active ingredients can be present in essentially more finely dispersed form than in the disperse phase of “macroemulsions” Another advantage is that they are sprayable as a result of their low viscosity. If microemulsions are used as cosmetics, corresponding products are characterized by high cosmetic elegance.

In particular, advantageous O/W microemulsions are those obtainable using phase-inversion temperature technology and then at least one emulsifier (emulsifier A), chosen from the group of emulsifiers having the following properties: their lipophilicity is dependent on the temperature in such a way that increasing the temperature increases the lipophilicity, and reducing the temperature decreases the lipophilicity of the emulsifier.

If the phase inversion is triggered essentially by varying the temperature, O/W emulsions, in particular O/W microemulsions, are obtainable, where the size of the oil droplets is essentially determined by the concentration of the emulsifier(s) used such that a higher emulsifier concentration results in relatively small droplets, and lower emulsifier concentration leads to relatively large droplets.

For the purposes of the present invention, it may be advantageous to use other W/O and/or O/W emulsifiers which do not fall under the definition of the emulsifier A, for example in order to increase the water resistance of the preparations according to the invention.

Further advantageous sprayable O/W emulsions for the purposes of the present invention are low-viscosity cosmetic or dermatological preparations of the oil-in-water type which comprise at least one oil phase and at least one water phase, where the preparation is stabilized by at least one gel former and must not necessarily comprise emulsifiers, but may comprise one or more emulsifiers.

Emulsions according to the invention for the purposes of the present invention, e.g. in the form of a skin protection cream, a skin lotion, a cosmetic milk, for example in the form of a sunscreen cream or a sun protection milk, are advantageous and comprise, for example, fats, oils, waxes and/or other fatty substances, and water and one or more emulsifiers as are customarily used for such a type of formulation.

Just as emulsions of liquid and solid consistency are used as cosmetic cleansing lotions or cleansing creams, the preparations according to the invention may also represent sprayable cleansing preparations (“cleansing sprays”), which are used, for example, for removing make-up or as mild washing lotion—optionally also for blemished skin. Such cleansing preparations can advantageously further be used as “rinse-off preparations”, which are rinsed off from the skin following application.

It is naturally known to the person skilled in the art that demanding cosmetic compositions are in most cases inconceivable without customary auxiliaries and additives. These include, for example, bodying agents, fillers, perfume, dyes, emulsifiers, additional active ingredients such as vitamins and proteins, light protection agents, stabilizers, insect repellents, alcohol, self-tanning substances, water, salts, antimicrobially, proteolytically or keratolytically active substances etc.

Correspsonding requirements apply mutates mutandis to the formulation of medicinal preparations.

Accordingly, cosmetic or topical dermatological preparations for the purposes of the present invention can, depending on the composition, be used, for example, as skin protection cream, cleansing milk, sun screen lotion, nourishing cream, day cream or night cream etc. It is in some instances possible and advantageous to use the compositions according to the invention as bases for pharmaceutical formulations.

It is likewise advantageous to use the preparations according to the invention for decorative cosmetics (make-up formulations).

Also favorable are those cosmetic and dermatological preparations which are in the form of a sun screen. In addition to the active ingredient used according to the invention, these preferably additionally comprise at least one broadband filter and/or at least one UVA filter substance and/or at least one UVB filter substance and/or at least one inorganic pigment.

It is, however, also advantageous for the purposes of the present invention to create cosmetic and dermatological preparations whose main purpose is not protection against sunlight, but which nevertheless have a content of UV protection substances. Thus, for example, UV-A and/or UV-B filter substances or broadband filters are usually incorporated into day creams.

Advantageous broadband filters, UV-A or UV-B filter substances are, for example, bis-resorcinyltriazine derivatives of the following structure:

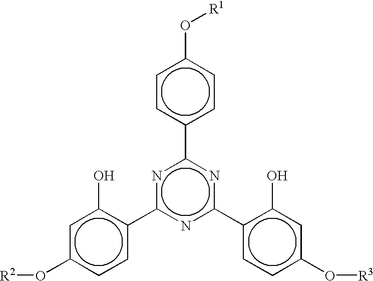

where R¹, R² and R³, independently of one another, are chosen from the group of branched and unbranched alkyl groups having 1 to 10 carbon atoms or represent a single hydrogen atom. Particular preference is given to 2,4-bis-{[4-(2-ethylhexyloxy)-2-hydroxy]phenyl}-6-(4-methoxyphenyl)-1,3,5-triazine (INCI: Aniso Triazine), which is available under the tradename Tinosorb® S from CIBA-Chemikalien GmbH.

Other UV filter substances which have the structural formula

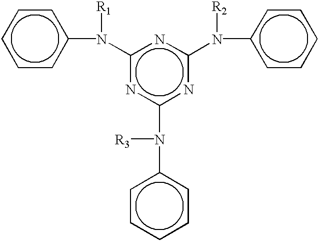

are advantageous UV filter substances for the purposes of the present invention, for example the s-triazine derivatives described in European Laid-Open Specification EP 570 838 A1, the chemical structure of which is given by the generic formula

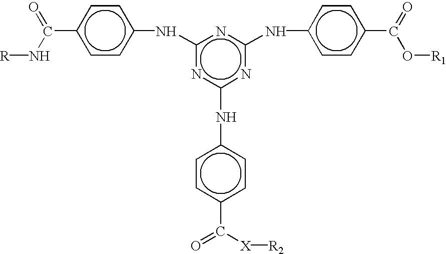

where

R is a branched or unbranched C₁-C₁₈-alkyl radical, a C₅-C₁₂-cycloalkyl radical, optionally substituted by one or more C₁-C₄-alkyl groups,

X is an oxygen atom or an NH group,

R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

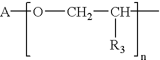

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

R₂ is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

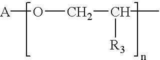

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

if X is an oxygen atom.

A particularly preferred UV filter substance for the purposes of the present invention is also an asymmetrically substituted s-triazine whose chemical structure is given by the formula

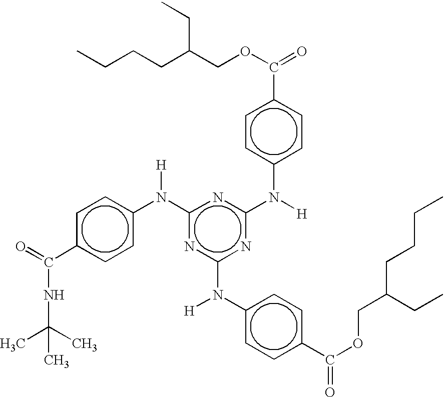

which is also referred to below as dioctylbutylamidotriazone (INCI: Diethylhexylbutamidotriazone) and is available under the tradename UVASORB® HEB from Sigma 3V.

Also advantageous for the purposes of the present invention is a symmetrically substituted s-triazine, tris(2-ethylhexyl) 4,4′,4″-(1,3,5-triazine-2,4,6-triyltriimino)trisbenzoate, synonym:

2,4,6-tris-[anilino(p-carbo-2′-ethyl-1′-hexyloxy)]-1,3,5-triazine (INCI: ethylhexyl triazone), which is sold by BASF Aktiengesellschaft under the tradename UVINUL® T 150.

European Laid-Open Specification 775 698 also describes preferred bis-resorcinyltriazine derivatives, the chemical structure of which is given by the generic formula

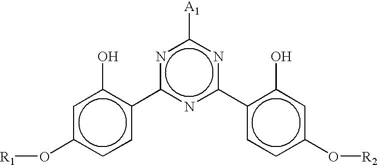

where R₁ and R₂ represent, inter alia, C₃-C₁₈-alkyl or C₂-C₁₈-alkenyl and Al is an aromatic radical.

Also advantageous for the purposes of the present invention are

2,4-bis{[4-(3-sulfonato)-2-hydroxypropyloxy)-2-hydroxy]phenyl}-6-(4-methoxyphenyl)-1,3,5-triazine sodium salt,

2,4-bis{[4-(3-(2-propyloxy)-2-hydroxypropyloxy)-2-hydroxy]phenyl}-6-(4-methoxyphenyl)-1,3,5-triazine,

2,4-bis{[4-(2-ethylhexyloxy)-2-hydroxy]phenyl}-6-[4-(2-methoxyethylcarboxyl)phenylamino]-1,3,5-triazine,

2,4-bis{[4-(3-(2-propyloxy)-2-hydroxypropyloxy)-2-hydroxy]phenyl}-6-[4-(2-ethylcarboxyl)phenylamino]-1,3,5-triazine,

2,4-bis{[4-(2-ethylhexyloxy)-2-hydroxy]phenyl}-6-(1-methylpyrrol-2-yl)-1,3,5-triazine,

2,4-bis{[4-tris(trimethyl-siloxysilylpropyloxy)-2-hydroxy]phenyl}-6-(4-methoxyphenyl)-1,3,5-triazine,

2,4-bis{[4-(2″-methylpropenyloxy)-2-hydroxy]phenyl}-6-(4-methoxyphenyl)-1,3,5-triazine and

2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

The total amount of one or more triazine derivatives optionally used in the finished cosmetic or dermatological preparations is advantageously chosen from the range 0.01% by weight to 15% by weight, preferably from 0.1 to 10% by weight, in each case based on the total weight of the preparations.

Advantageous sulfonated, water-soluble UV filters for the purposes of the present invention are:

Phenylene-1,4-bis(2-benzimidazyl)-3,3′-5,5′-tetrasulfonic acid, which is characterized by the following structure:

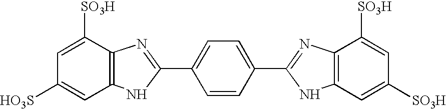

and its salts, particularly the corresponding sodium, potassium or triethanolammonium salts, in particular the phenylene-1,4-bis(2-benzimidazyl)-3,3′-5,5′-tetrasulfonic acid bis-sodium salt

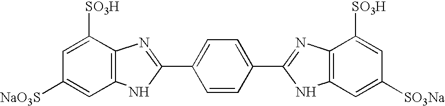

with the INCI name bisimidazylate (CAS No.: 180898-37-7), which is available, for example, under the tradename Neo Heliopan® AP from Haarmann & Reimer.

A further advantageous sulfonated UV filter for the purposes of the present invention are the salts of 2-phenylbenzimidazol-5-sulfonic acid, and its sodium, potassium or its triethanolammonium salt, and the sulfonic acid itself

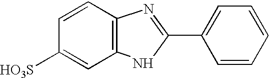

with the INCI name Phenylbenzimidazole Sulfonic Acid (CAS No. 27503-81-7), which is available, for example, under the tradename Eusolex® 232 from Merck or under Neo Heliopan® Hydro from Haarmann & Reimer.

A further advantageous sulfonated UV filter is 3,3′-(1,4-phenylenedimethylene) bis(7,7-dimethyl-2-oxo-bicyclo-[2.2.1]hept-1-ylmethanesulfonic acid, and its sodium, potassium or its triethanolammonium salt, and the sulfonic acid itself:

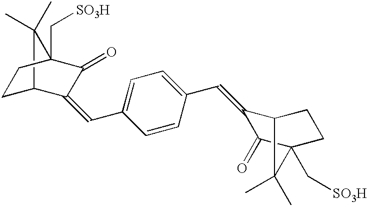

with the INCI name Terephthalidene Dicamphor Sulfonic Acid (CAS No.: 90457-82-2), which is available, for example, under the tradename Mexoryl® SX from Chimex.

Further advantageous water-soluble UV-B and/or broadband filter substances are, for example:

Sulfonic acid derivatives of 3-benzylidenecamphors, such as, for example, 4-(2-oxo-3-bornylidenemethyl)benzenesulfonic acid, 2-methyl-5-(2-oxo-3-bornylidenemethyl)sulfonic acid and salts thereof.

The total amount of one or more sulfonated UV filter substances optionally used in the finished cosmetic or dermatological preparations is advantageously chosen from the range 0.01% by weight to 20% by weight, preferably from 0.1 to 10% by weight, in each case based on the total weight of the preparations.

The UV-B and/or broadband filters may be oil-soluble or water-soluble. Advantageous oil-soluble UV-B and/or broadband filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate, polyethoxyethyl 4-bis (polyethoxy)amino benzoate (available under the tradename Uvinul® P25 from BASF);

Derivatives of benzophenone, preferably 2-hydroxy-4-methoxybenzophenone (available under the tradename Uvinul® M40 from BASF), 2-hydroxy-4-methoxy-4′-methylbenzophenone, 2,2′-dihydroxy-4-methoxybenzophenone, 2-hydroxy-4-methoxybenzophenone-5-sulfonic acid (available under the tradename Uvinul® MS40 from BASF);

and UV filters bonded to polymers.

Particularly advantageous UV filter substances which are liquid at room temperature for the purposes of the present invention are homomenthyl salicylate, 2-ethylhexyl 2-cyano-3,3-diphenylacrylate, 2-ethylhexyl-2-hydroxybenzoate and esters of cinnamic acid, preferably 2-ethylhexyl 4-methoxycinnamate and isopentyl 4-methoxycinnamate.

Homomenthyl salicylate (INCI: Homosalate) is characterized by the following structure:

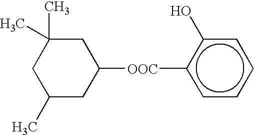

2-ethylhexyl 2-cyano-3,3-diphenylacrylate (INCI: Octocrylene) is available from BASF under the name Uvinul® N 539T and is characterized by the following structure:

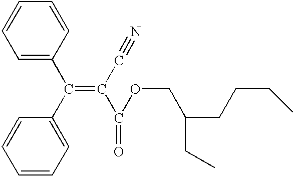

2-ethylhexyl 2-hydroxybenzoate (2-ethylhexyl salicylate, octyl salicylate, INCI: ethylhexyl salicylate) is available, for example, from Haarmann & Reimer under the tradename Neo Heliopan® OS and is characterized by the following structure:

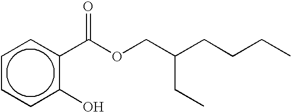

2-ethylhexyl 4-methoxycinnamate (INCI: Ethylhexyl Methoxycinnamate) is available, for example, from BASF under the tradename Uvinul® MC 80 and is characterized by the following structure:

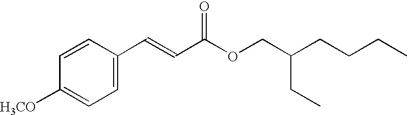

Isopentyl 4-methoxycinnamate (INCI: Isoamyl p-Methoxycinnamate) is available, for example, from Haarmann & Reimer under the tradename Neo Heliopan® E 1000 and is characterized by the following structure:

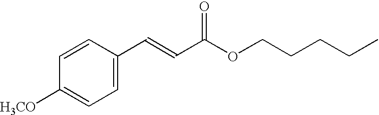

Another advantageous UV filter substance which is liquid at room temperature for the purposes of the present invention is (3-(4-(2,2-bisethoxycarbonylvinyl)phenoxy)propenyl)methylsiloxane/dimethylsiloxane copolymer, which is available, for example, from Hoffmann-La Roche under the tradename Parsol® SLX.

The total amount of one or more UV filter substances liquid at room temperature optionally used in the finished cosmetic or dermatological preparations is advantageously chosen from the range 0.1% by weight to 30% by weight, preferably from 0.5 to 20% by weight, in each case based on the total weight of the preparations.

Advantageous dibenzoylmethane derivatives for the purposes of the present invention are, in particular, 4-(tert-butyl)-4′-methoxydibenzoylmethane (CAS No. 70356-09-1), which is sold by BASF under the name Uvinul® BMBM and by Merck under the tradename Eusolex® 9020 and is characterized by the following structure:

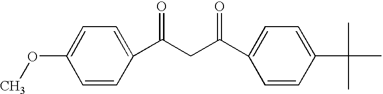

A further advantageous dibenzoylmethane derivative is 4-isopropyldibenzoylmethane (CAS No. 63250-25-9), which is sold by Merck under the name Eusolex® 8020. Eusolex 8020 is characterized by the following structure:

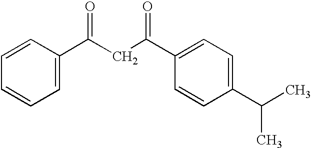

benzotriazoles are characterized by the following structural formula:

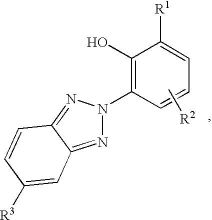

in which

R¹ and R², independently of one another, are linear or branched, saturated or unsaturated, substituted (e.g. substituted by a phenyl radical) or unsubstituted alkyl radicals having 1 to 18 carbon atoms and/or polymer radicals which themselves do not absorb UV rays (such as, for example, silicone radicals, acrylate radicals and the like), and R₃ is chosen from the group H or alkyl radical having 1 to 18 carbon atoms.

An advantageous benzotriazole for the purposes of the present invention is 2,2′-methylenebis(6-(2H-benzotriazol-2-yl)-4-(1,1,3,3-tetramethylbutyl)phenol), a broadband filter which is characterized by the chemical structural formula

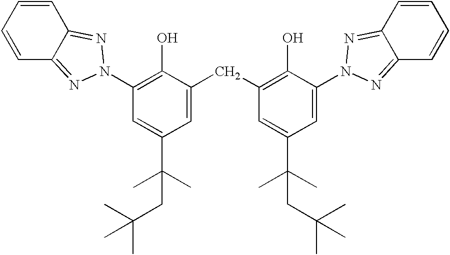

and is available under the tradename Tinosorb® M from CIBA-Chemikalien GmbH.

An advantageous benzotriazole for the purposes of the present invention is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane, which is sold by Chimex under the name Mexoryl® XL and is characterized by the following chemical structural formula

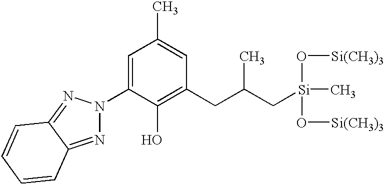

Further advantageous benzotriazoles for the purposes of the present invention are [2,4′-dihydroxy-3-(2H-benzotriazol-2-yl)-5-(1,1,3,3-tetramethylbutyl)-2′-n-octoxy-5′-benzoyl]diphenylmethane, 2,2′-methylenebis[(6-(2H-benzotriazol-2-yl)-4-(methyl)phenol], 2,2′-methylenebis[6-(2H-benzotriazol-2-yl)-4-(1,1,3,3-tetramethylbutyl)phenol], 2-(2′-hydroxy-5′-octylphenyl)benzotriazole, 2-(2′-hydroxy-3′,5′-di-t-amylphenyl)benzotriazole and 2-(2′-hydroxy-5′-methylphenyl)benzotriazole.

The total amount of one or more benzotriazoles optionally used in the finished cosmetic or dermatological preparations is advantageously chosen from the range 0.1 to 20% by weight, preferably from 0.5 to 15% by weight, very particularly preferably from 0.5 to 10% by weight, in each case based on the total weight of the preparations.

Another UV-A filter advantageous for the purposes of the present invention is the 2-(4-ethoxyanilinomethylene)propanedicarboxylic diethyl ester, described in EP-A-0 895 776, of the following formula.

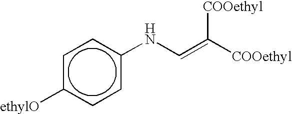

Cosmetic and dermatological preparations according to the invention also advantageously, but not obligatorily, comprise inorganic pigments based on metal oxides and/or other metal compounds which are insoluble or virtually insoluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals and mixtures of such oxides. These pigments are X-ray amorphous or non-X-ray amorphous. The pigments are articularly preferably based on TiO2.

X-ray amorphous oxide pigments are metal oxides or semimetal oxides which reveal no or no recognizable crystal structure in X-ray diffraction experiments. Such pigments are often obtainable by flame reaction, for example by reacting a metal or semimetal halide with a hydrogen and air (or pure oxygen) in a flame.

In cosmetic, dermatological or pharmaceutical formulations, X-ray amorphous oxide pigments are used as thickeners and thixotropic agents, flow auxiliaries, for emulsion and dispersion stabilization and as carrier substance (for example for increasing volume of finely divided powders).

X-ray amorphous oxide pigments which are known and are often used in cosmetic or dermatological technology are the silicon oxides of the Aerosil® type (CAS No. 7631-86-9). Aerosils®, obtainable from DEGUSSA, are characterized by low particle size (e.g. between 5 and 40 nm), where the particles are to be regarded as spherical particles of very uniform dimension. Macroscopically, Aerosile® are recognizable as loose, white powders. Within the meaning of the present invention, X-ray amorphous silicon dioxide pigments are particularly advantageous and, of these, precisely those of the Aerosil® grade are preferred.

Advantageous Aerosil® grades are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974, Aerosil® R976.

The total amount of one or more X-ray amorphous oxide pigments optionally used in the finished cosmetic or dermatological preparations is advantageously chosen from the range 0.1 to 20% by weight, preferably from 0.5 to 10% by weight, very particularly preferably from 1 to 5% by weight, in each case based on the total weight of the preparations.

According to the invention, the non-X-ray-amorphous inorganic pigments are advantageously in hydrophobic form, i.e. they have been surface-treated to repel water. This surface treatment may involve providing the pigments with a thin hydrophobic layer by methods known per se.

Such a process consists, for example, in producing the hydrophobic surface layer according to a reaction as in

nTiO₂+m(RO)3Si—R′→nTiO₂ (surf.).

Here, n and m are stoichiometric parameters to be used as desired, and R and R′ are the desired organic radicals. Hydrophobicized pigments prepared as in DE-A 33 14 742, for example, are advantageous.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel), octyltrimethoxysilane or alginic acid. These organic surface coatings may be present alone, in combination and/or in combination with inorganic coating materials.

Zinc oxide particles suitable according to the invention and predispersions of zinc oxide particles are available under the following tradenames from the companies listed:

| Tradename | Coating | Manufacturer |
|---|---|---|
| Z-Cote ® HP1 | 2% Dimethicone | BASF |
| Z-Cote ® | — | BASF |
| ZnO NDM | 5% Dimethicone | H&R |
| MZ-505 S | 5% Methicone | Tayca Corp. |

Suitable titanium dioxide particles and predispersions of titanium dioxide particles are available under the following tradenames from the companies listed:

| Tradename | Coating | Manufacturer |
|---|---|---|
| MT-100TV | Aluminum | Tayca Corporation |
| | hydroxide/stearic acid | |
| MT-100Z | Aluminum | Tayca Corporation |
| | hydroxide/stearic acid | |
| Eusolex ® T-2000 | Alumina/simethicone | Merck KgaA |
| Titanium dioxide | Octyltrimethoxysilane | Degussa, BASF |
| T805 (Uvinul ® | | |
| TiO₂) | | |

Advantageous TiO₂ pigments are available, for example, under the tradename Uvinul® TiO₂, and advantageous TiO₂/Fe₂O₃ mixed oxides are available under the tradename Uvinul® TiO₂ A from BASF.

The total amount of inorganic pigments, in particular hydrophobic inorganic micropigments, optionally used in the finished cosmetic or dermatological preparations is advantageously chosen from the range from 0.1 to 30% by weight, preferably 0.1 to 10.0, in particular 0.5 to 6.0% by weight, based on the total weight of the preparations.

The cosmetic and dermatological preparations according to the invention can comprise cosmetic active ingredients, auxiliaries and/or additives, as are customarily used in such preparations, e.g. antioxidants, preservatives, bacteriocides, perfumes, antifoams, dyes, pigments which have a coloring action, thickeners, surface-active substances, emulsifiers, emollients, moisturizers and/or humectants, fats, oils, waxes or other customary constituents of a cosmetic or dermatological formulation, such as alcohols, polyols, polymers, foam stabilizers, electrolytes, organic solvents or silicone derivatives.

Preservatives permitted in food technology, which are listed below with their E number, are to be used advantageously according to the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | p-hydroxybenzoic ethyl |
| | ester Na salt |
| E 216 | n-propyl p-hydroxybenzoate |
| E 217 | p-hydroxybenzoic-n-propyl |
| | ester Na salt |
| E 218 | methyl p-hydroxybenzoate |
| E 219 | p-hydroxybenzoic methyl |
| | ester Na salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogen sulfite |
| E 228 | Potassium hydrogen |
| | sulfite |
| E 230 | Biphenyl (Diphenyl) |
| E 231 | Orthophenylphenol |
| E 232 | Sodium |
| | orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also suitable are preservatives or preservative auxiliaries customary in cosmetics: dibromodicyanobutane (2-bromo-2-bromomethylglutarodinitrile), 3-iodo-2-propinylbutylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol.+Formaldehyde donors.

Also suitable as preservatives are phenyl hydroxyalkyl ethers, in particular the compounds known under the name phenoxyethanol, because of their bactericidal and fungicidal effects on a number of microorganisms.

Other antimicrobial agents are likewise suitable for being incorporated into the preparations according to the invention. Advantageous substances are, for example, 2,4,4′-trichloro-2′-hydroxydiphenyl ether (Irgasan), 1,6-di(4-chlorophenylbiguanido)hexane (Chlorhexidin), 3,4,4′-trichlorocarbanilide, quaternary ammonium compounds, oil of cloves, mint oil, thyme oil, triethyl citrate, farnesol (3,7,11-trimethyl-2,6,10-dodecatrien-1-ol) and the active ingredients or active ingredient combinations described in the patent laid-open specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160,

DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium hydrogencarbonate can also be used advantageously.

For the purposes of the present invention, it is, moreover, advantageous to add other anti-irritative or anti-inflammatory active ingredients to the preparations, in particular batyl alcohol (a-octadecyl glyceryl ether), selachyl alcohol (α-9-octadecenyl glyceryl ether), chimyl alcohol (α-hexadecyl glyceryl ether), bisabolol and/or panthenol.

It is likewise advantageous to add conventional antioxidants to the preparations for the purposes of the present invention. According to the invention, favorable antioxidants can be any antioxidants which are suitable or customary for cosmetic and/or dermatological applications.

The antioxidants are advantageously selected from the group consisting of amino acids (for example glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, ψ-lycopene) and derivatives thereof, chlorogenic acid and derivatives thereof, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very small tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of antioxidants (one or more compounds) in the preparations is preferably from 0.001 to 30% by weight, particularly preferably 0.05-20% by weight, in particular 1-10% by weight, based on the total weight of the preparation.

If vitamin E and/or derivatives thereof are used as the antioxidant(s), it is advantageous to choose their respective concentrations from the range 0.001-10% by weight, based on the total weight of the formulation.

Preparations according to the present invention can also be used as a basis for cosmetic or dermatological deodorants or antiperspirants. All active ingredients which are common for deodorants or antiperspirants can be used advantageously, for example odor maskers such as the customary perfume constituents, odor absorbers, for example the phyllosilicates described in patent laid-open specification DE-P 40 09 347, and of these, in particular montmorillonite, kaolinite, illite, beidellite, nontronite, saponite, hectorite, bentonite, smectite, and also, for example, zinc salts of ricinoleic acid.

The amount of such active ingredients (one or more compounds) in the preparations according to the invention is preferably 0.001 to 30% by weight, particularly preferably 0.05 to 20% by weight, in particular 1 to 10% by weight, based on the total weight of the preparation.

The water phase of the cosmetic preparations for the purposes of the present invention may also have gel character which, in addition to an effective content of substances used according to the invention and solvents customarily used therefor, preferably comprises water, further organic and/or inorganic thickeners and/or hydrocolloids.

The inorganic thickener(s) can, for example, advantageously be chosen from the group of modified or unmodified, naturally occurring or synthetic phyllosilicates.

Although it is entirely favorable to use pure components, it may, however, be advantageous to incorporate mixtures of different modified and/or unmodified phyllosilicates into compositions according to the invention.

For the purposes of this replication, phyllosilicates are understood as meaning silicates and alumosilicates in which the silicate or aluminate units are linked together via three Si—O— or Al—O— bonds and form a wavy sheet or layer structure. The fourth Si—O— or Al—O— valence is saturated by cations. Relatively weak electrostatic interactions, e.g. hydrogen bridge bonds, exist between the individual layers. The layer structure, meanwhile, is largely defined by strong, covalent bonds.

The stochiometry of the sheet silicates is (Si₂O₅²⁻) for pure silicate structures and (AlₘSi²⁻ₘO₅^{(2+m)-)} for alumosilicates.

m is a number greater than zero and less than 2.

If pure silicates are not present, but alumosilicates, the circumstance that each Si⁴⁺ group replaced by Al³⁺ requires another singly charged cation to neutralize the charge is to be taken into account.

The charge balance is preferably evened out by H⁺, alkali metal ions or alkaline earth metal ions. Aluminum as counterion is also known and advantageous. In contrast to the alumosilicates, these compounds are called aluminum silicates. “Aluminum alumosilicates”, in which aluminum is present both in the silicate network, and also as counterion, are known and sometimes advantageous for the present invention.

Phyllosilicates are well documented in the literature, e.g. in the “Lehrbuch der Anorganischen Chemie” [Textbook of Inorganic Chemistry], A. F. Hollemann, E. Wiberg and N. Wiberg, 91st-100th Ed., Walter de Gruyter—Verlag 1985, passim, and “Lehrbuch der Anorganischen Chemie”, H. Remy, 12th Ed., Akademische Verlagsgesellschaft, Leipzig 1965, passim. The layer structure of montmorillonite is given in Römpps Chemie-Lexikon, Franckh'sche Verlagshandlung W. Keller & Co., Stuttgart, 8th Ed., 1985, p. 2668 f.

Examples of phyllosilicates are:

| | |
|---|---|
| Montmorillonite | Na_{0.33}((Al_{1.67}Mg_{0.33}) (OH)₂(Sᵢ₄O₁₀)) |
| often simplified: | Al₂O₃*4SiO₂*H₂O*nH₂O or |
| | Al₂[(OH)₂/Si₄O₁₀].n H₂O |
| Kaolinite | Al₂(OH)₄(Si₂O₅) |
| Illite | (K,H₃O)_{y}(Mg₃(OH)₂(Si_{4-y}Al_{y}O₁₀)) or |
| | (K,H₃O)_{y}(Al₂(OH)₂(Si_{4-y}Al_{y}O₁₀)) |
| | where y = 0.7 − 0.9 |
| Beidellite | (Ca,Na)_{0.3}(Al₂(OH)₂(Al_{0.5}Si_{3.5}O₁₀)) |
| Nontronite | Na_{0.33}(Fe₂(OH)₂(Al_{0.33}S_{i3.67}O₁₀)) |
| Saponite | (Ca,Na)_{0.33}((Mg,Fe)₃(OH)₂(Al_{0.33}Si_{3.67}O₁₀)) |
| Hectorite | Na_{0.33}((Mg,Li)₃(OH,F)₂(Si₄O₁₀)) |

Montmorillonite is the main mineral of the naturally occurring bentonites.

Very advantageous inorganic gel formers for the purposes of the present invention are aluminum silicates, such as the montmorillonites (bentonites, hectorites and derivatives thereof, such as quaternium-18 bentonite, quaternium-18 hectorites, stearalkonium bentonites and stearalkonium hectorites), and also magnesium-aluminum silicates (Veegum® grades), and sodium-magnesium silicates (Laponite® grades).

Montmorillonites represent clay minerals which are a type of dioctahedral smectites, and are masses which swell in water, but do not become plastic. The layer packets in the three-layer structure of the montmorillonites can swell as a result of reversible incorporation of water (in a 2- to 7-fold amount) and other substances, such as, for example, alcohols, glycols, pyridine, α-picoline, ammonium compounds, hydroxy-aluminosilicate ions etc.

The chemical formula given above is only approximate: since montmorillonites have a large capacity for ion exchange, Al can be replaced by Mg, Fe²⁺, Fe³⁺, Zn, Pb (e.g. from harmful substances in waste waters), Cr, and also Cu and others. The resulting negative charge of the octahedral layers is compensated by cations, in particular Na⁺ (sodium montmorillonite) and Ca²⁺ (calcium montmorillonite is only swellable to a very small extent) in interlayer positions.

Synthetic magnesium silicates and/or bentonites advantageous for the purposes of the present invention are sold, for example, by Süd-Chemie under the tradename Optigel®.

An advantageous aluminum silicate advantageous for the purposes of the present invention is sold, for example, by R. T. Vanderbilt Comp., Inc., under the tradename Veegum®. The various Veegum® grades, which are all advantageous according to the invention, are characterized by the following compositions

| | (regular | | | | |
|---|---|---|---|---|---|
| | grade) | HV | K | HS | S-728 |
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| Ashing loss | 11.1 | 12.6 | 7.6 | 5.5 | 7.5 |

These products swell in water to form viscous gels, which have an alkaline reaction. The organophilization of montmorillonite or bentonites (exchange of the interlayer cations for quaternary alkylammonium ions) produces products (bentones) which are preferably used for dispersion in organic solvents and oils, fats, ointments, inks, surface coatings and in detergents.

Bentone® is a tradename for various neutral and chemically inert gelling agents which are constructed from long-chain, organic ammonium salts and specific types of montmorillonite. Bentones swell in organic media and cause the latter to swell. The gels are resistant in diluted acids and alkalis, although they partially lose their gelling properties upon prolonged contact with strong acids and alkalis. Because of their organophilic character, the bentones are only wettable by water with difficulty.

The following Bentone® grades are sold, for example, by Kronos Titan: Bentone® 27, an organically modified montmorillonite, Bentone® 34 (dimethyldioctylammonium bentonite), which is prepared in accordance with U.S. Pat. No. 2,531,427 and, because of its lipophilic groups, swells more readily in lipophilic medium than in water, Bentone® 38, an organically modified montmorillonite, a cream-colored to white powder, Bentone® LT, a purified clay mineral, Bentone® Gel MIO, an organically modified montmorillonite which is supplied as a very fine suspension in mineral oil (SUS-71) (10% bentonite, 86.7% mineral oil and 3.3% wetting agent), Bentone® Gel IPM, an organically modified bentonite which is suspended in isopropyl myristate (10% bentonite, 86.7% isopropylmyristate, 3.3% wetting agent), Bentone® Gel CAO, an organically modified montmorillonite which is taken up in castor oil (10% bentonite, 86.7% castor oil and 3.3% wetting agent), Bentone® Gel Lantrol, an organically modified montmorillonite which, in paste form, is intended for the further processing, in particular for the preparation, of cosmetic compositions; 10% bentonite, 64.9 Lantrol (wool wax oil), 22.0 isopropyl myristate, 3.0 wetting agent and 0.1 propyl p-hydroxybenzoate, Bentone® Gel Lan I, a 10% strength Bentone® 27 paste in a mixture of wool wax USP and isopropyl palmitate, Bentone® Gel Lan II, a bentonite paste in pure liquid wool wax, Bentone® Gel NV, a 15% strength Bentone® 27 paste in dibutyl phthalate, Bentone® Gel OMS, a bentonite paste in Shellsol T., Bentone® Gel OMS 25, a bentonite paste in isoparaffinic hydrocarbons (Idopar® H), Bentone® Gel IPP, a bentonite paste in isopropyl palmitate.

“Hydrocolloid” is the technological abbreviation for the more correct name “hydrophilic colloid”. Hydrocolloids are macromolecules which have a largely linear structure and have intermolecular forces of interaction which permit secondary and primary valence bonds between the individual molecules and thus the formation of a recticular structure. Some are water-soluble natural or synthetic polymers which, in aqueous systems, form gels or viscous solutions. They increase the viscosity of the water by either binding water molecules (hydration) or else by absorbing and encapsulating the water into their interwoven macromolecules, at the same time as restricting the mobility of the water. Such water-soluble polymers represent a large group of chemically very different natural and synthetic polymers whose common feature is their solubility in water or aqueous media. A prerequisite for this is that these polymers have a number of hydrophilic groups sufficient for solubility in water and are not too greatly crosslinked.

The hydrophilic groups can be nonionic, anionic or cationic in nature, for example as follows:

| | | | | |
|---|---|---|---|---|
| —NH₂—NH—R —OH | —COOH 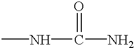 | —COO⁻—SO₃⁻—PO₃²⁻ | M⁺M⁺M²⁺ | 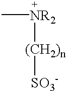 |
| —SH —O— 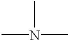 | 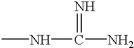 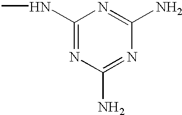 | 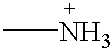 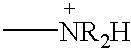 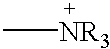 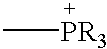 | X⁻X⁻X⁻X⁻ | 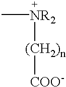 |

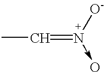

The group of the cosmetically and dermatologically relevant hydrocolloids can be divided as follows into:

organic, natural compounds, such as, for example, agar agar, carrageen, tragacanth, gum arabic, alginates, pectins, polyoses, guar flour, carob bean flour, starch, dextrins, gelatins, casein;

organic, modified natural substances, such as, for example, carboxymethylcellulose and other cellulose ethers, hydroxyethylcellulose and hydroxypropylcellulose and microcristalline cellulose;

organic, completely synthetic compounds, such as, for example, polyacrylic and polymethacrylic compounds, vinyl polymers, polycarboxylic acids, polyethers, polyimines, polyamides, polyurethanes;

inorganic compounds, such as, for example, polysilicic acids, clay minerals, such as montmorillonites, zeolites, silicas.

An advantageous stabilizer are ethylcelluloses. Ethylcelluloses are characterized by the following structure:

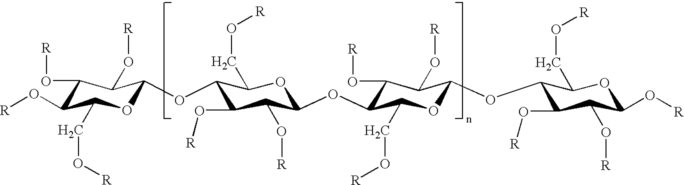

Here, R can either be ethyl groups or hydrogen atoms.

The degree of ethylation in the ethylcellulose is advantageously 2.0 to 3.0, corresponding to 40 to 55%, preferably 48.0 to 49.5% ethylation. The average molecular mass is preferably to be chosen such that the viscosity of a 5% strength solution in a mixture of 80 parts of toluene and 20 parts of ethanol at 25° C. is 3 to 110 mPas, preferably 9 to 11 mPas. The average molar mass is particularly advantageously 100,000 to 400,000 g/mol.

The content of ethylcellulose in the preparations according to the invention is preferably 0.1 to 10% by weight, based on the total weight of the preparations. Such products are available, for example, under the tradename ETHOCEL® Standard 10 Premium (Dow Chemicals).

Microcristalline cellulose is an advantageous hydrocolloid for the purposes of the present invention. It is available, for example, from “FMC Corporation Food and Pharmaceutical Products” under the tradename Avicel®. A particularly advantageous product for the purposes of the present invention is the grade Avicel® RC-591, which is a modified microcristalline cellulose which is made up of 89% microcrystalline cellulose and 11% sodium carboxymethylcellulose. Other commercial products from this class of raw material are Avicel® RC/CL, Avicel® CE-15, Avicel® 500.

Further hydrocolloids used advantageously are, for example, methylcelluloses, which is the name for the methylethers of cellulose. They are characterized by the following structural formula

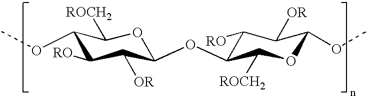

in which R can be a hydrogen or a methyl group.

Particularly advantageous for the purposes of the present invention are the cellulose mixed ethers, which are generally likewise referred to as methylcelluloses, which contain, in addition to a predominating content of methyl groups, also 2-hydroxyethyl, 2-hydroxypropyl or 2-hydroxybutyl groups. Particular preference is given to (hydroxypropyl)methylcelluloses, for example those available under the tradename Methocel® E4M from Dow Chemical Comp.

Also advantageous according to the invention is sodium carboxymethylcellulose, the sodium salt of the glycolic ether of cellulose, for which R in the above structural formula may be hydrogen and/or CH₂—COONa. Particular preference is given to sodium carboxymethylcellulose available under the tradename Natrosol® Plus 330 CS from Aqualon and also referred to as cellulose gum.

Also preferred for the purposes of the present invention is xanthan (CAS No. 11138-66-2), also called xanthan gum, which is an anionic heteropolysaccharide which is generally formed by fermentation from maize sugar and is isolated as potassium salt. It is produced by xanthomonas campestris and some other species under aerobic conditions and has a molecular weight of from 2×10⁶ to 24×10⁶. Xanthan is formed from a chain having β-1,4-bonded glucose (cellulose) with side chains. The structure of the subgroups consists of glucose, mannose, glucuronic acid, acetate and pyruvate. The number of pyruvate units determines the viscosity of the xanthan.

A further advantageous gel former for the purposes of the present invention is also carrageen, a gel-forming extract with a similar structure to agar, of North Atlantic red algae which belong to the Florideae (Chondrus crispus and Gigartina stellata).

The term carrageen is frequently used for the dried algae product and carrageenan for the extract thereof. The carrageen precipitated from the hot-water extract of the algae is a colorless to sand-colored powder with a molecular weight range from 100,000 to 800,000 and a sulfate content of about 25%. Carrageen, which is very readily soluble in warm water, forms a thixotropic gel upon cooling, even if the water content is 95-98%. The rigidity of the gel is effected by the double helix structure of the carrageen.

In the case of carrageenan, three principle constituents are differentiated: The gel-forming κ fraction consists of D-galactose 4-sulfate and 3,6-anhydro-α-D-galactose, which has alternate glycoside bonds in the 1,3- and 1,4 position (by contrast, agar contains 3,6-anhydro-α-L-galactose). The nongelling λ fraction is composed of 1,3-glycosidically linked D-galactose 2-sulfate and 1,4-bonded D-galactose-2,6-disulfate radicals, and is readily soluble in cold water. ι-Carrageenan, composed of D-galactose 4-sulfate in 1,3 bond and 3,6-anhydro-α-D-galactose 2-sulfate in 1,4 bond, is both water-soluble and also gel-forming. The nature of cations which are present (K⁺, NH₄⁺, Na⁺, Mg²⁺, Ca²⁺) also influences the solubility of the carrageens.

The use of chitosan in cosmetic preparations is known per se. Chitosan represents a partially deacylated chitin. This biopolymer has, inter alia, film-forming properties and is characterized by a silky feel on the skin. A disadvantage, however, is its severe stickiness on the skin which occurs in particular—temporarily—during application. In individual cases corresponding preparations may not then be marketable since they are unacceptable to and/or viewed negatively by the consumer. As is known, chitosan is used, for example, in hair care. It is suitable, to a better degree than the chitin on which it is based, as a thickener or stabilizer and improves the adhesion and water resistance of polymeric films. A representative of a large number of literature references for the prior art is: H. P. Fiedler, “Lexikon der Hilfsstoffe fur Pharmazie, Kosmetik und angrenzende Gebiete” [Lexikon of auxiliaries for pharmacy, cosmetics and related fields], third edition 1989, Editio Cantor, Aulendorf, p. 293, keyword “Chitosan”.

Chitosan is characterized by the following structural formula:

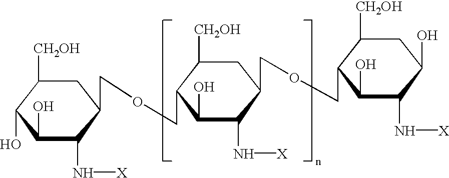

where n assumes values up to about 10 000, and X is either the acetyl radical or hydrogen. Chitosan forms by deacetylation and partial depolymerization (hydrolysis) of chitin, which is characterized by the structural formula

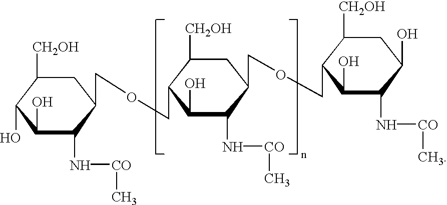

Chitin is an essential constituent of the ecto skeleton [′oχιτωv=Greek: integument] of arthropods (e.g. insects, crabs, spiders) and is also found in supporting tissues of other organisms (e.g. molluscs, algae, fungi).

In the region of about pH <6, chitosan is positively charged and in that range is also soluble in aqueous systems. It is incomPatible with anionic raw materials. For this reason, to prepare chitosan-containing oil-in-water emulsions, the use of nonionic emulsifiers is appropriate. These are known per se, for example from EP-A 776 657.

Preference is given according to the invention to chitosans with a degree of deacetylation of >25% , in particular >55 to 99% [determined by means of ¹H-NMR].

It is advantageous to choose chitosans with molecular weights between 10 000 and 1 000 000, in particular those with molecular weights between 100 000 and 1 000 000 [determined by means of gel permeation chromatography].

Polyacrylates are gelling agents likewise to be used preferably for the purposes of the present invention. Polyacrylates advantageous according to the invention are acrylate-alkyl acrylate copolymers, in particular those chosen from the group of carbomers or carbopols (Carbopol® is actually a registered trademark of B. F. Goodrich ComPany). In particular, the acrylate-alkyl acrylate copolymers advantageous according to the invention are characterized by the following structure:

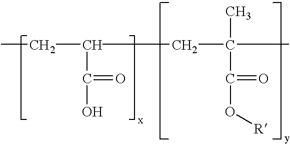

where R′ is a long-chain alkyl radical, and x and y represent numbers which symbolize the respective stoichiometric proportion of each of the comonomers.

According to the invention, particular preference is given to acrylate copolymers and/or acrylate-alkyl acrylate copolymers which are available under the trade names Carbopol® 1382, Carbopol® 981 and Carbopol® 5984 from B. F. Goodrich ComPany and very particular preference is give to polyacrylates from the group of Carbopol grades 980, 981, 1382, 2984, 5984 and Carbomer 2001.

Also advantageous are copolymers of C₁₀-₃₀-alkyl acrylates and one or more monomers of acrylic acid, of methacrylic acid or esters thereof which are crosslinked with an allyl ether of sucrose or an allyl ether of pentaerythritol.

Compounds which carry the INCI name “Acrylates/C₁₀-₃₀ Alkyl Acrylate Crosspolymer” are likewise advantageous. Particularly advantageous are those polymers available under the trade names Pemulen® TR1 and Pemulen® TR2 from B. F. Goodrich ComPany.

Compounds which carry the INCI name ammonium acryloyldimethyltaurates/vinylpyrrolidone copolymers are advantageous.

According to the invention, the ammonium acryloyldimethyl taurate/vinylpyrrolidone copolymers have the empirical formula [C₇H₁₆N₂SO₄]ₙ [C₆H₉NO]ₘ, which corresponds to the following statistical structure

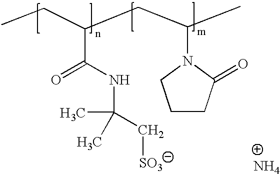

Preferred species for the purposes of the present invention are listed in the Chemical Abstracts under the Registry numbers 58374-69-9, 13162-05-5 and 88-12-0 and are available under the tradename Aristoflex® AVC from Clariant GmbH.

Also advantageous are copolymers/crosspolymers comprising Acryloyldimethyl Taurate, such as, for example, Simugel® EG or Simugel® EG from Seppic S.A.

Further completely synthetic hydrocolloids to be used according to the invention are:

A. anionic polyurethanes which are soluble or dispersible in water and which are advantageously obtainable from

Aa) at least one compound which contains two or more active hydrogen atoms per molecule,

Ab) at least one diol containing acid or salt groups and

Ac) at least one diisocyanate.

The component Aa) is, in particular, a diol, aminoalcohol, diamine, polyesterol, polyetherol with a number-average molecular weight of in each case up to 3000, or mixtures thereof, where up to 3 mol % of said compounds may be replaced by triols or triamines. Preference is given to diols and polyesterdiols. In particular, the component Aa) comprises at least 50% by weight, based on the total weight of the component Aa), of a polyesterdiol. Suitable polyesterdiols are all those which are customarily used for the preparation of polyurethanes, in particular the reaction products of phthalic acid and diethylene glycol, isophthalic acid and 1,4-butanediol, isophthalic acid/adipic acid and 1,6-hexanediol, and adipic acid and ethylene glycol or 5-NaSO₃-isophthalic acid, phthalic acid, adipic acid and 1,6-hexanediol.

Examples of diols which can be used are ethylene glycol, propylene glycol, butylene glycol, neopentyl glycol, polyetherols, such as polyethylene glycols having molecular weights up to 3000, block copolymers of ethylene oxide and propylene oxide with number-average molecular weights of up to 3000 or block copolymers of ethylene oxide, propylene oxide and butylene oxide which contain the copolymerized alkylene oxide units in randomly distributed manner or in the form of blocks. Preference is given to ethylene glycol, neopentyl glycol, di-, tri-, tetra-, penta- or hexaethylene glycol. Other diols which can be used are poly(α-hydroxycarboxylic acid)diols.

Suitable aminoalcohols are, for example, 2-aminoethanol, 2-(N-methylamino)ethanol, 3-aminopropanol or 4-aminobutanol.

Exmaples of suitable diamines are ethylenediamine, propylenediamine, 1,4-diaminobutane and 1,6-diaminohexane, and α,ω-diamines which can be prepared by amination of polyalkylene oxides with ammonia.

Component Ab) is, in particular, dimethylolpropanoic acid or compounds of the formulae

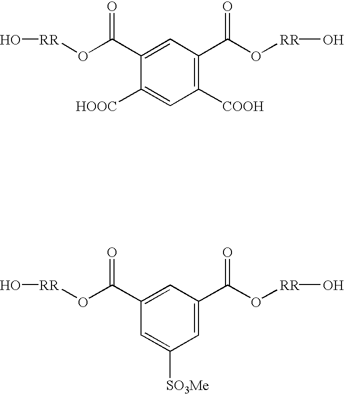

where RR is in each case a C₂-C₁₈-alkylene group and Me is Na or K.

Component Ac) is, in particular, hexamethylene diisocyanate, isophorone diisocyanate, methyldiphenyl isocyanate (MDI) and/or tolylene diisocyanate.

The polyurethanes are obtainable by reacting the compounds of groups Aa) and Ab) under an inert-gas atmosphere in an inert solvent at temperatures of from 70 to 130° C. with the compounds of group Ac). This reaction can be carried out, where appropriate, in the presence of chain extenders in order to prepare polyurethanes with relatively high molecular weights. As is customary in the preparation of polyurethanes, the components [(Aa)+(Ab)]:Ac are advantageously used in the molar ratio of from 0.8 to 1.1:1. The acid number of the polyurethanes is determined by the composition and the concentration of the compounds of component (Ab) in the mixture of components (Aa) and (Ab).

The polyurethanes have K values according to H. Fikentscher (determined in 0.1% strength by weight solutions in N-methylpyrrolidone at 25° C. and pH 7) of from 15 to 100, preferably 25 to 50.

The K value, also referred to as the intrinsic viscosity, is a parameter which is easy to determine by means of viscosity measurements of polymer solutions and is therefore frequently used in the industrial sector for characterizing polymers.

The polyurethanes containing acid groups are, after neutralization (partial or complete), water-soluble or dispersible without the aid of emulsifiers. The salts of the polyurethanes generally have better solubility or dispersibility in water than the unneutralized polyurethanes. Bases which can be used for the neutralization of the polyurethanes are alkali metal bases, such as sodium hydroxide solution, potassium hydroxide solution, soda, sodium hydrogencarbonate, potassium carbonate or potassium hydrogen carbonate and alkaline earth metal bases, such as calcium hydroxide, calcium oxide, magnesium hydroxide or magnesium carbonate, and ammonia and amines. 2-Amino-2-methylpropanol, diethylaminopropylamine and triisoproanolamine have proven particularly useful for the neutralization of the polyurethanes containing acid groups. The neutralization of the polyurethanes containing acid groups can also be carried out using mixtures of two or more bases, e.g. mixtures of sodium hydroxide solution and triisopropanolamine. Depending on the intended use, neutralization may be partial, e.g. 20 to 40%, or complete, i.e. 100%.

These polymers and their preparation are described in more detail in DE-A-42 25 045, to the entire scope of which reference is hereby made.

B. Water-soluble or -dispersible cationic polyurethanes and polyureas of

Ba) at least one diisocyanate, which may have already been reacted beforehand with one or more compounds which contain two or more active hydrogen atoms per molecule, and

Bb) at least one diol, primary or secondary amino alcohol, primary or secondary diamine or primary or secondary triamine with one or more tertiary, quaternary or protonated tertiary amino nitrogen atoms.

Preferred diisocyanates are as given above under A). Compounds with two or more active hydrogen atoms are diols, aminoalcohols, diamines, polyesterols, polyamidediamines and polyetherols. Suitable compounds of this type are as given above under A).

The polyurethanes are prepared as described above under A). Charged cationic groups can be produced in the polyureas from the tertiary amino nitrogen atoms present either by protonation, e.g. with carboxylic acids, such as lactic acid, or by quaternization, e.g. with alkylating agents, such as C₁ to C₄-alkyl halides or sulfates. Examples of such alkylating agents are ethyl chloride, ethyl bromide, methyl chloride, methyl bromide, dimethyl sulfate and diethyl sulfate.

These polymers and their preparation are described in more detail in DE-A-42 41 118, to the entire scope of which reference is hereby made.

C. Linear polyurethanes with carboxylate groups of

Ca) a 2,2-hydroxymethyl-substituted carboxylic acid of the formula

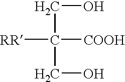

in which RR′ is a hydrogen atom or a C₁-C₂₀-alkyl group, which is used in an amount which suffices for 0.35 to 2.25 milliequivalents of carboxyl groups to be present in the polyurethane per g of polyurethane,

Cb) 10 to 90% by weight, based on the weight of the polyurethane, of one or more organic compounds with not more than two active hydrogen atoms and

Cc) one or more organic diisocyanates.

The carboxyl groups present in the polyurethane are, finally, at least partially neutralized with a suitable base. These polymers and their preparation are described in EP-A-619 111, to the entire scope of which reference is hereby made.

D. Carboxyl-containing polycondensation products of anhydrides of tri- or tetracarboxylic acids and diols, diamines or aminoalcohols (polyesters, polyamides or polyester amides). These polymers and their preparation are described in more detail in DE-A-42 24 761, to the entire scope of which reference is hereby made.

E. Polyacrylates and polymethacrylates, as are described in more detail in DE-A-43 14 305, 36 27 970 and 29 17 504. Reference is hereby made to these publications in their entirety.

The polymers used according to the invention preferably have a K value of from 15 to 100, preferably 25 to 50. The polymers are generally present in the composition according to the invention in an amount in the range from 0.2 to 20% by weight, based on the total weight of the compositions. The salt is used in an amount effective for improving the exchangeability of the polymers. The salt is generally used in an amount of from 0.02 to 10% by weight, preferably 0.05 to 5% by weight and in particular 0.1 to 3% by weight, based on the total weight of the composition.

The total amount of one or more hydrocolloids in the finished cosmetic or dermatological preparations is advantageously chosen to be less than 5% by weight, preferably between 0.05 and 3.0% by eight, particularly preferably between 0.1 and 1.0% by weight, based on the total weight of the preparations.

In addition, it may be advantageous to add interface- or surface-active agents to preparations according to the invention, for example cationic emulsifiers such as, in particular, quaternary surfactants.

Quaternary surfactants contain at least one N atom which is covalently bonded to 4 alkyl or aryl groups. This leads, irrespective of the pH, to a positive charge. Alkylbetain, alkylamidopropylbetain and alkylamidopropylhydroxysultaine are advantageous. The cationic surfactants used according to the invention may also preferably be chosen from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds with a cationic character, such as amine oxides, for example alkyldimethylamine oxides or alkylaminoethyldimethylamine oxides. In particular, cetyltrimethylammonium salts are to be used advantageously.

It is also advantageous to use cationic polymers (e.g. Jaguar® C 162 [Hydroxypropyl Guar Hydroxypropyltrimonium Chloride] or modified magnesium aluminum silicates (e.g. quaternium-18-hectorite, which is available, for example, under the tradename Bentone® 38 from Rheox, or stearalkonium hectorite, which is available, for example, under the tradename Softisan® gel from Hüls AG).

Preparations according to the invention can advantageously also comprise oil thickeners in order to improve the tactile properties of the emulsions and the stick consistency. Advantageous oil thickeners for the purposes of the present invention are, for example, other solids, such as, for example, hydrophobic silicon oxides of the Aerosil® type, which are available from Degussa AG. Examples of advantageous Aerosil® grades are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974 and/or Aerosil® R976.

In addition, “metal soaps” (i.e. salts of higher fatty acids with the exception of alkali metal salts) are also advantageous oil thickeners for the purposes of the present invention, such as, for example, aluminum stearate, zinc stearate and/or magnesium stearate.

It is likewise advantageous to add amphoteric or zwitterionic surfactants (e.g. cocamidopropylbetain) and moisturizers (e.g. betain) to preparations according to the invention. Examples of amphoteric surfactants to be used advantageously are acyl/dialkylethylenediamine, for example sodium acylamphoacetate, disodium acylamphodipropionate, disodium alkylamphodiacetate, sodium acylamphohydroxypropylsulfonate, disodium acylamphodiacetate and sodium acylamphopropionate, N-alkylamino acids, for example aminopropylalkylglutamide, alkylaminopropionic acid, sodium alkylimidodipropionate and lauroamphocarboxyglycinate.

The amount of surface- or interface-active substances (one or more compounds) in the preparations according to the invention is preferably 0.001 to 30% by weight, particularly preferably 0.05 to 20% by weight, in particular 1 to 10% by weight, based on the total weight of the preparation.

A surprising property of the preparations according to the invention is that they are very good vehicles for cosmetic or dermatological active ingredients into the skin, preferred active ingredients being the aforementioned antioxidants, which can protect the skin against oxidative stress.

According to the invention, the active ingredients (one or more compounds) can also very advantageously be chosen from the group of lipophilic active ingredients, in particular from the following group:

Acetylsalicylic acid, atropine, azulene, hydrocortisone and derivatives thereof, e.g. hydrocortisone-17-valerate, vitamins of the B and D series, very favorably vitamin B₁, vitamin B₁₂, vitamin D₁, but also bisabolol, unsaturated fatty acids, namely the essential fatty acids (often also called vitamin F), in particular γ-linolenic acid, oleic acid, eicosapentenoic acid, docosahexenoic acid and derivatives thereof, chloramphenicol, caffeine, prostaglandins, thymol, camphor, extracts or other products of a vegetable and animal origin, e.g. evening primrose oil, borrage oil or currant seed oil, fish oils, cod-liver oil and also ceramides and ceramide-like compounds, etc.

It is also advantageous to choose the active ingredients from the group of refatting substances, for example purcellin oil, Eucerit® and Neocerit®.

The active ingredient(s) is/are particularly advantageously chosen from the group of NO synthesase inhibitors, particularly if the preparations according to the invention are to be used for the treatment and prophylaxis of the symptoms of intrinsic and/or extrinsic skin aging and for the treatment and prophylaxis of the harmful effects of ultraviolet radiation on the skin.

A preferred NO synthase inhibitor is nitroarginine.

The active ingredient(s) is/are further advantageously chosen from the group which includes catechins and bile esters of catechins and aqueous or organic extracts from plants or sections of plants which have a content of catechins or bile esters of catechins, such as, for example, the leaves of the Theaceae plant family, in particular of the species Camellia sinensis (green tea). Their typical ingredients (such as, for example, polyphenols or catechins, caffeine, vitamins, sugars, minerals, aminoacids, lipids) are particularly advantageous.

Catechins are a group of compounds which are to be regarded as hydrogenated flavones or anthocyanidines and are derivatives of “catechin” (catechol, 3,3′,4′,5,7-flavanpentol, 2-(3,4-dihydroxyphenyl)chroman-3,5,7-triol). Epicatechin ((2R,3R)-3,3′,4′,5,7-flavanpentol) is also an advantageous active ingredient for the purposes of the present invention.

Also advantageous are plant extracts with a content of catechin, in particular extracts of green tea, such as, for example, extracts from leaves of the plants of the species Camellia spec., very particularly the tea types Camellia sinenis, C. assamica, C. taliensis and C. irrawadiensis and hybrids of these with, for example, Camellia japonica.

Preferred active ingredients are also polyphenols and catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (also often collectively called “flavones”) are also advantageous active ingredients for the purposes of the present invention. They are characterized by the following basic structure (substitution positions are shown):

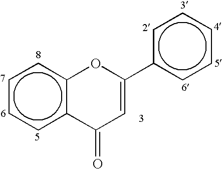

Some of the more important flavones which can also preferably be used in preparations according to the invention are given in table 2 below:

TABLE 2
OH substitution positions

| | 3 | 5 | 7 | 8 | 2′ | 3′ | 4′ | 5′ |
|---|---|---|---|---|---|---|---|---|
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually in glycosylated form.

According to the invention, the flavonoids are preferably chosen from the group of substances of the generic structural formula

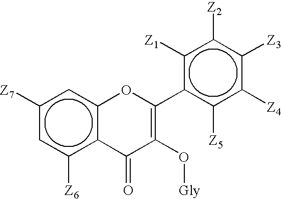

where Z₁ to Z₇, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono- and oligoglycoside radicals.

According to the invention, the flavonoids can, however, also advantageously be chosen from the group of substances of the generic structural formula

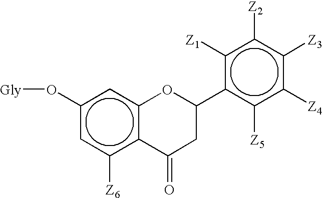

where Z₁ to Z₆, independently of one another, are chosen from the group consisting of H, OH, alkoxy and hydroxyalkoxy, where the alkoxy and hydroxyalkoxy groups may be branched or unbranched and have 1 to 18 carbon atoms, where Gly is chosen from the group mono and oligoglycoside radicals.

Preferably, such structures can be chosen from the group of substances of the generic structural formula

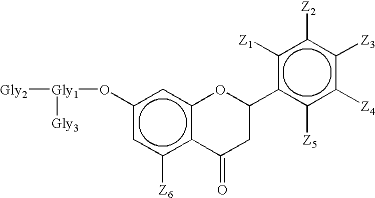

where Gly₁, Gly₂ and Gly₃, independently of one another, are monoglycoside radicals. Gly₂ and Gly₃ may also, individually or together, represent saturations by hydrogen atoms.

Preferably, Gly₁, Gly₂ and Gly₃, independently of one another, are chosen from the group of hexosyl radicals, in particular the rhamnosyl radicals and glucosyl radicals. However, hexosyl radicals, for example allosyl, altrosyl, galactosyl, gulosyl, idosyl, mannosyl and talosyl, can also be used advantageously in some circumstances. It may also be advantageous according to the invention to use pentosyl radicals.

Z₁ to Z₅ are, independently of one another, advantageously chosen from the group consisting of H, OH, methoxy, ethoxy and 2-hydroxyethoxy, and the flavone glycosides have the structure

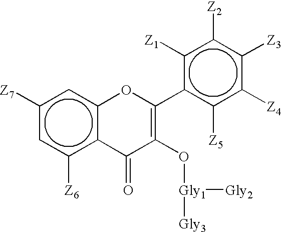

The flavone glycosides according to the invention are particularly advantageously chosen from the group given by the following structure:

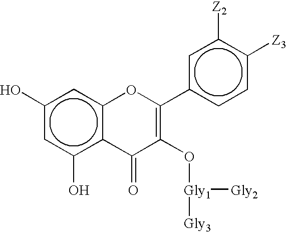

where Gly₁, Gly₂ and Gly₃, independently of one another, are monoglycoside radicals. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also advantageously be used in some circumstances. It may also be advantageous according to the invention to use pentosyl radicals.

For the purposes of the present invention, it is particularly advantageous to choose the flavone glucoside(s) from the group consisting of α-glucosylrutin, α-glucosylmyricetin, α-glucosylisoquercitrin, α-glucosylisoquercetin and α-glucosylquercitrin.

Particular preference is given according to the invention to α-glucosylrutin.

Also advantageous according to the invention are naringin (aurantin, naringenin-7-rhamno-glucoside), hesperidin 3′,5,7-trihydroxy-4′-methoxyflavanone-7-rutinoside, hesperidoside, hesperetin-7-O-rutinoside), rutin (3,3′,4′,5,7-pentahydroxyflavone-3-rutinoside, quercetin-3-rutinoside, sophorin, birutan, rutabion, taurutin, phytomelin, melin), troxerutin (3,5-dihydroxy-3′,4′,7-tris(2-hydroxyethoxy)flavone-3-(6-O-(6-deoxy-α-L-mannopyranosyl)-β-D-glucopyranoside)), monoxerutin (3,3′,4′,5-tetrahydroxy-7-(2-hydroxyethoxy)flavone-3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxyflavanone), eriodictyol-7-glucoside (3′,4′,5,7-tetrahydroxyflavanone-7 glucoside), flavanomarein (3′,4′,7,8-tetrahydroxyflavanone-7 glucoside) and isoquercetin (3,3′,4′,5,7-pentahydroxyflavanone-3-(β-D-glucopyranoside). It is also advantageous to choose the active ingredient(s) from the group of ubiquinones and plastoquinones.

Ubiquinones are characterized by the structural formula

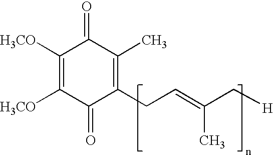

and are the most widespread and thus the most investigated bioquinones. Ubiquinones are referred to, depending on the number of isoprene units linked in the side chain, as Q-1, Q-2, Q-3 etc., or according to the number of carbon atoms, as U-5, U-10, U-15 etc. They preferably arise with certain chain lengths, e.g. in some microorganisms and yeasts where n=6. In most mammals including man, Q10 predominates.

Coenzyme Q10 is particularly advantageous and is characterized by the following structural formula:

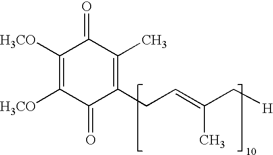

Plastoquinones have the general structural formula

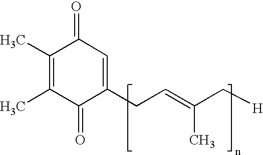

Plastoquinones differ in the number n of isoprene radicals and are referred to accordingly, e.g. PQ-9 (n=9). In addition, other plastoquinones with varying substituents on the quinone ring exist.

Creatine and/or creatine derivatives are preferred active ingredients for the purposes of the present invention. Creatine is characterized by the following structure:

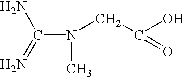

Preferred derivatives are creatine phosphate and creatine sulfate, creatine acetate, creatine ascorbate and the derivatives esterified at the carboxyl group with mono- or polyfunctional alcohols.

A further advantageous active ingredient is L-carnitine [3-hydroxy-4-(trimethylammonio)butyrobetaine]. Acylcarnitines, chosen from the group of substances of the following general structural formula

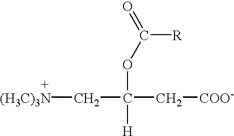

where R is chosen from the group of branched and unbranched alkyl radicals having up to 10 carbon atoms, are advantageous active ingredients for the purposes of the present invention. Preference is given to propionylcarnitine and, in particular, acetylcarnitine. Both enantiomers (D and L form) are to be used advantageously for the purposes of the present invention. It may also be advantageous to use any enantiomer mixtures, for example a racemate of D and L form.

Further advantageous active ingredients are sericoside, pyridoxol, vitamin K and biotin and aroma substances.

The list of said active ingredients and active ingredient combinations which can be used in the preparations according to the invention is, of course, not intended to be limiting. The active ingredients can be used individually or in any combinations with one another.

The amount of such active ingredients (one or more compounds) in the preparations according to the invention is preferably 0.001 to 30% by weight, particularly preferably 0.05 to 20% by weight, in particular 0.1 to 10% by weight, based on the total weight of the preparation.

In some instances, it may also be advantageous for the purposes of the present invention to incorporate dyes and/or color pigments into the preparations according to the invention.

The dyes and color pigments can be chosen from the corresponding positive list of the Cosmetics Directive or the EC list of cosmetic colorants. In most cases they are identical to the dyes approved for foods. Advantageous color pigments are, for example, titanium dioxide, mica, iron oxides (e.g. Fe₂O₃, Fe₃O₄, FeO(OH)) and/or tin oxide. Advantageous dyes are, for example, carmine, Berlin blue, chrome oxide green, ultramarine blue and/or manganese violet. It is particularly advantageous to choose the yes and/or color pigments from the following list. The Colour Index Numbers (CIN) are taken from the Rowe Colour Index, 3rd Edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxy- | 12085 | red |
| naphthalene | | |
| Pigment Red 3 | 12120 | red |
| Ceres red; Sudan red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfodiethylamido-1′-phenyla- | 12490 | red |
| zo)-3-hydroxy-5″-chloro-2″,4″-dimethoxy-2-naph- | | |
| thanilide | | |
| Disperse Yellow 16 | 12700 | yellow |
| 1-(4-Sulfo-1-phenylazo)-4-aminobenzene-5-sul- | 13015 | yellow |
| fonic acid | | |
| 2,4-Dihydroxyazobenzene-4′-sulfonic acid | 14270 | orange |
| 2-(2,4-Dimethylphenylazo-5-sulfo)-1-hydroxy- | 14700 | red |
| naphthalene-4-sulfonic acid | | |
| 2-(4-Sulfo-1-naphthylazo)-1-naphthol-4-sulfonic | 14720 | red |
| acid | | |
| 2-(6-Sulfo-2,4-xylylazo)-1-naphthol-5-sulfonic | 14815 | red |
| acid | | |
| 1-(4′-Sulfophenylazo)-2-hydroxynaphthalene | 15510 | orange |
| 1-(2-Sulfo-4-chloro-5-carboxy-1-phenyla- | 15525 | red |
| zo)-2-hydroxynaphthalene | | |
| 1-(3-Methylphenylazo-4-sulfo)-2-hydroxynaphtha- | 15580 | red |
| lene | | |
| 1-(4′,(8′)-Sulfonaphthylazo)-2-hydroxynaphtha- | 15620 | red |
| lene | | |
| 2-Hydroxy-1,2′-azonaphthalene-1′-sulfonic acid | 15630 | red |
| 3-Hydroxy-4-phenylazo-2-naphthylcarboxylic | 15800 | red |
| acid | | |
| 1-(2-Sulfo-4-methyl-1-phenylazo)-2-naphthyl- | 15850 | red |
| carboxylic acid | | |
| 1-(2-Sulfo-4-methyl-5-chloro-1-phenylazo)-2-hy- | 15865 | red |
| droxynaphthalene-3-carboxylic acid | | |
| 1-(2-Sulfo-1-naphthylazo)-2-hydroxynaphtha- | 15880 | red |
| lene-3-carboxylic acid | | |
| 1-(3-Sulfo-1-phenylazo)-2-naphthol-6-sulfonic | 15980 | orange |
| acid | | |
| 1-(4-Sulfo-1-phenylazo)-2-naphthol-6-sulfonic | 15985 | yellow |
| acid | | |
| Allura Red | 16035 | red |
| 1-(4-Sulfo-1-naphthylazo)-2-naphthol-3,6-disul- | 16185 | red |
| fonic acid | | |
| Acid Orange 10 | 16230 | orange |
| 1-(4-Sulfo-1-naphthylazo)-2-naphthol-6,8-disul- | 16255 | red |
| fonic acid | | |
| 1-(4-Sulfo-1-naphthylazo)-2-naphthol-3,6,8-tri- | 16290 | red |
| sulfonic acid | | |
| 8-Amino-2-phenylazo-1-naphthol-3,6-disulfonic | 17200 | red |
| acid | | |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hy- | 19140 | yellow |
| droxy-pyrazolone-3-carboxylic acid | | |
| Pigment Yellow 16 | 20040 | yellow |
| 2,6-(4′-Sulfo-2″,4″-dimethyl)bisphenyla- | 20170 | orange |
| zo)-1,3-dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naph- | 27755 | black |
| thylazo]-1-hydroxy-7-aminonaphthalene-3,6-di- | | |
| sulfonic acid | | |
| 4′-[(4″-Sulfo-1″-phenylazo)-7′-sulfo-1′-naph- | 28440 | black |
| thylazo]-1-hydroxy-8-acetylaminonaphtha- | | |
| lene-3,5-disulfonic acid | | |
| Direct Orange 34, 39, 44, 46, 60 | 40215 | orange |
| Food Yellow | 40800 | orange |
| trans-β-Apo-8′-carotinaldehyde (C₃₀) | 40820 | orange |
| trans-Apo-8′-carotinic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(diethylami- | 42051 | blue |
| no)triphenylcarbinol | | |
| 4-[(4-N-Ethyl-p-sulfobenzylamino)phenyl(4-hy- | 42053 | green |
| droxy-2-sulfophenyl)(methylene)-1-(N-ethyl- | | |
| N-p-sulfobenzyl)-2,5-cyclohexadienimine] | | |
| Acid Blue 7 | 42080 | blue |
| (N-Ethyl-p-sulfobenzylamino)phenyl(2-sulfophe- | 42090 | blue |
| nyl)methylene-(N-ethyl-N-p-sulfobenzyl)Δ^{2,5}-cy- | | |
| clohexadienimine | | |
| Acid Green 9 | 42100 | green |
| Diethyldisulfobenzyldi-4-amino-2-chloro- | 42170 | green |
| di-2-methyl-fuchsonimmonium | | |
| Basic Violet 14 | 42510 | violet |
| Basic Violet 2 | 42520 | violet |
| 2′-Methyl-4′-(N-ethyl-N-m-sulfobenzyl)ami- | 42735 | blue |
| no-4″-(N-diethyl)amino-2-methyl-N-ethyl- | | |
| N-m-sulfobenzyl fuchsonimmonium | | |
| 4′-(N-Dimethyl)amino-4″-(N-phenyl)aminonaphtho- | 44045 | blue |
| N-dimethyl-fuchsonimmonium | | |
| 2-Hydroxy-3,6-disulfo-4,4′-bisdimethylamino- | 44090 | green |
| naphtho-fuchsonimmonium | | |
| Acid Red 52 | 45100 | red |
| 3-(2′-Methylphenylamino)-6-(2′-methyl-4′-sulfo- | 45190 | violet |
| phenylamino)-9-(2″-carboxyphenyl)xanthenium | | |
| salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluo- | 45410 | red |
| rescein | | |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum com- | 58000 | red |
| plex | | |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis (o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinone azine | 69800 | blue |
| Vat Blue 6; Pigment Blue 64 | 69825 | blue |
| Vat Orange 7 | 71105 | orange |
| Indigo | 73000 | blue |
| Indigo-disulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated Phthalocyanines | 74260 | green |
| Natural Yellow 6,19; Natural Red 1 | 75100 | yellow |
| Bixin, Nor-Bixin | 75120 | orange |
| Lycopene | 75125 | yellow |
| trans-alpha-, beta- and gamma-carotene | 75130 | orange |
| Keto- and/or hydroxyl derivates of carotene | 75135 | yellow |
| Guanine or pearlizing agent | 75170 | white |
| 1,7-Bis(4-hydroxy-3-methoxyphenyl)-1,6-hepta- | 75300 | yellow |
| diene-3,5-dione | | |
| Complex salt (Na, Al, Ca) of carminic acid | 75470 | red |
| Chlorophyll a and b; copper compounds of chlo- | 75810 | green |
| rophylls and Chlorophyllins | | |
| Aluminum | 77000 | white |
| Hydrated alumina | 77002 | white |
| Hydrous aluminum silicates | 77004 | white |
| Ultramarine | 77007 | blue |
| Pigment Red 101 und 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268 | black |
| Chromium oxide | 77288 | green |
| Chromium oxide, hydrous | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide, hydrated | 77492 | yellow |
| Iron oxide | 77499 | black |
| Mixtures of iron (II) and iron (III) hexacyano- | 77510 | blue |
| ferrate | | |
| Pigment White 18 | 77713 | white |
| Manganese ammonium diphosphate | 77742 | violet |
| Manganese phosphate; Mn₃(PO₄)₂.7 H2O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)-isoalloxazine, | | yellow |
| lactoflavine | | |
| Sugar coloring | | brown |
| Capsanthin, capsorubin | | orange |
| Betanin | | red |
| Benzopyrylium salts, Anthocyans | | red |
| Aluminum, zinc, magnesium and calcium stearate | | white |
| Bromothymol blue | | blue |
| Bromocresol green | | green |
| Acid Red 195 | | red |

It may also be favorable to choose one or more substances from the following group as the dye: 2,4-dihydroxyazobenzene, 1-(2′-chloro-4′-nitro-1′-phenylazo)-2-hydroxynaphthalene, Ceres Red, 2-(4-sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1- (2-sulfo-1-naphthylazo)-2-hydroxynaphthalene-3-carboxylic acid, aluminum salt of l-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5,6′-tetrachloro-2,4,5,7-tetrabromofluorescein and its aluminum salt, aluminum salt of 2,4,5,7-tetraiodofluorescein, aluminum salt of quinophthalone disulfonic acid, aluminum salt of indigo disulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, β-carotene or cochenille.

Also advantageous for the purposes of the present invention are gel creams with a content of pearlescent pigments. Preference is given in particular to the types of pearlescent pigments listed below:

Natural pearlescent pigments, such as, for example

“pearl essence” (guanine/hypoxanthin mixed crystals from fish scales), “mother of pearl” (ground mussel shells) and monocrystalline pearlescent pigments, such as, for example, bismuth oxychloride (BiOCl)

layer substrate pigments: e.g. mica/metal oxide

Bases for pearlescent pigments are, for example, pulverulent pigments or castor oil dispersions of bismuth oxychloride and/or titanium dioxide, and bismuth oxychloride and/or titanium dioxide on mica. The luster pigment listed under CIN 77163, for example, is particularly advantageous.

Also advantageous are, for example, the following types of pearlescent pigment based on mica/metal oxide:

| | Coating/layer | |
|---|---|---|
| Group | thickness | Color |
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold |
| | | shades |
| | TiO₂/Cr₂O₃ | green |
| | TiO₂/Berlin blue | deep blue |
| | TiO₂/carmine | red |

Particular preference is given, for example, to the pearlescent pigments available from Merck under the trade names Timiron, Colorona or Dichrona.

The list of given pearlescent pigments is not of course intended to be limiting. Pearlescent pigments which are advantageous for the purposes of the present invention are obtainable in numerous ways known per se. For example, other substrates apart from mica can be coated with further metal oxides, such as, for example, silica and the like. SiO₂ particles coated with, for example, TiO₂ and Fe₂O₃ (“ronaspheres”), which are sold by Merck and are particularly suitable for the optical reduction of fine lines are advantageous.

It can moreover be advantageous to dispense completely with a substrate such as mica. Particular preference is given to pearlescent pigments prepared using SiO₂. Such pigments, which may also additionally have goniochromatic effects, are available, for example, under the trade name Sicopearl Fantastico from BASF.

Pigments from Engelhard/Mearl based on calcium sodium borosilicate which have been coated with titanium dioxide can also be used advantageously. These are available under the name Reflecks. In addition to the color, as a result of their particle size of from 40 nm to 180 μm, they have a glitter effect.

In addition, also particularly advantageous are effect pigments which are available under the trade name Metasomes Standard/Glitter in various colors (yellow, red, green, blue) from Flora Tech. The glitter particles are present here in the mixtures with various auxiliaries and dyes (such as, for example, the dyes with the Colour Index (CI) Numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from e.g. 0.1% by weight to 30% by weight, preferably from 0.5 to 15% by weight, in particular from 1.0 to 10% by weight, in each case based on the total weight of the preparations.

The invention also provides a process for the preparation of the preparations according to the invention, which comprises combining and heating the constituents of the oil phase or of the water phase separately, and then combining them together with stirring and, particularly advantageously, with homogenization, very particularly advantageously with stirring with moderate to high input of energy, advantageously using a gear rim dispersing machine at a rotary number up to at most 10000 rpm, preferably from 2500 to 7700 rpm.

The present invention will be illustrated in more detail by reference to the examples below (formulation recipes).

### EXAMPLES 1 TO 3

O/W Emulsions

| | Ex. 1 | Ex. 2 | Ex. 3 |
|---|---|---|---|
| Glyceryl stearate citrate | 2.00 | 1.50 | 3.00 |
| Glyceryl stearate SE | | | |
| Glyceryl stearate | | | |
| PEG-40 Stearate | | | |
| Stearic acid | | | |
| Caprylic/capric triglyceride | 0.50 | | 1.00 |
| Cetearyl alcohol | 3.00 | 1.00 | |
| Cetyl alcohol | | | 3.00 |
| Stearyl alcohol | | 1.00 | |
| Lanolin alcohol | | 0.50 | |
| Myristyl alcohol | | | |
| Octyldodecanol | 1.50 | | |
| Mineral oil | | | 2.00 |
| Compound I | 0.50 | 2.00 | 0.50 |
| Dimethicone diethylbenzalmalonate | | | |
| Ethylhexyl methoxycinnamate | 6.50 | 5.00 | |
| Bis-ethylhexyloxyphenol methoxyphenyl | 2.50 | 2.00 | 1.50 |
| triazine | | | |
| Butyl methoxydibenzoylmethane | 1.10 | | 1.00 |
| Phenyl dibenzimidazoletetrasulfonic acid | | | 0.50 |
| Ethylhexyltriazone | 2.20 | | |
| 4-Methylbenzylidene camphor | | | |
| Octocrylene | | 5.00 | |
| Diethylhexylbutamidotriazone | 1.00 | 3.00 | |
| Phenylbenzimidazolsulfonic acid | | | |
| Methylenebis-benzotriazolyltetramethyl- | | | 2.00 |
| butylphenol | | | |
| Drometrizol trisiloxane | | | |
| Terephthalidenedicamphorsulfonic acid | | | |
| Benzophenone-3 | | | |
| Ethylhexyl salicylate | | | |
| Homosalate | | | |
| Isoamyl p-methoxycinnamate | | | |
| Titanium dioxide aluminum hydroxide/ | 2.00 | 1.50 | 3.00 |
| stearic acid coating | | | |
| Zinc oxide HP1 | | | 1.00 |
| C₁₂—C₁₅-alkyl benzoate | 2.00 | | 1.50 |
| Dicaprylyl carbonate | | 3.00 | |
| Butylene glycol dicaprylate/dicaprate | 5.00 | | |
| Butylene glycol | | 7.50 | |
| Carbomer | 0.30 | 0.10 | 0.20 |
| Dicaprylyl ether | 2.50 | 0.50 | |
| PVP/Hexadecene copolymer | 1.00 | | 0.80 |
| Xanthan gum | 0.20 | | 0.40 |
| Dimethicone | 2.00 | | |
| Cyclomethicone | | 2.50 | |
| Tocopheryl acetate | 1.00 | | |
| Glycerol | 5.00 | | 7.50 |
| Dye (water- and/or oil-soluble) | | | 0.25 |
| DMDM hydantoin | | | 0.20 |
| Propylparaben | | 0.40 | |
| Methylparaben | 0.60 | | |
| Ethanol | | | |
| Konkaben LMB ® | | | |
| Trisodium EDTA | 1.00 | | 1.00 |
| Phenoxyethanol | 0.15 | | 0.30 |
| Perfume | q.s. | q.s. | q.s. |
| Aqua | Ad 100 | Ad 100 | Ad 100 |

### EXAMPLES 4 TO 6

O/E Emulsions

| | Ex. 4 | Ex. 5 | Ex. 6 |
|---|---|---|---|
| Glyceryl stearate citrate | | | |
| Glyceryl stearate SE | | 2.00 | |
| Glyceryl stearate | 2.00 | | 2.00 |
| PEG-40 Stearate | | | 2.00 |
| Stearic acid | 3.00 | 2.00 | |
| Caprylic/capric triglyceride | | | 1.50 |
| Cetearyl alcohol | | 2.00 | |
| Cetyl alcohol | | | 2.00 |
| Stearyl alcohol | | 0.50 | 2.00 |
| Lanolin alcohol | 0.50 | 0.50 | |
| Myristyl alcohol | 3.00 | 2.00 | |
| Octyldodecanol | | | |
| Mineral oil | | | |
| Compound I | 5.00 | 4.00 | 3.50 |
| Dimethicone diethylbenzalmalonate | 5.00 | | |
| Ethylhexyl methoxycinnamate | | | 10.00 |
| Bis-ethylhexyloxyphenol methoxyphenyl | | | 1.00 |
| triazine | | | |
| Butyl methoxydibenzoylmethane | | 2.00 | |
| Phenyl dibenzimidazoletetrasulfonic acid | 2.00 | | |
| Ethylhexyltriazone | | | |
| 4-Methylbenzylidene camphor | | 2.00 | |
| Octocrylene | | | 3.50 |
| Diethylhexylbutamidotriazone | | 1.50 | |
| Phenylbenzimidazolesulfonic acid | 1.00 | | 2.00 |
| Methylenebis-benzotriazolyltetramethyl- | | | |
| butylphenol | | | |
| Drometrizol Trisiloxane | | | 2.00 |
| Terephthalidenedicamphorsulfonic acid | | | 0.50 |
| Benzophenone-3 | 2.00 | | |
| Ethylhexyl salicylate | | 5.00 | |
| Homosalate | 3.00 | | |
| Isoamyl p-methoxycinnamate | | 1.00 | |
| Titanium dioxide aluminum hydroxide/ | | | 1.00 |
| stearic acid coating | | | |
| Zinc oxide HP1 | | | |
| C₁₂—C₁₅-alkyl benzoate | 2.50 | | 0.80 |
| Dicaprylyl carbonate | | 2.00 | |
| Butylene glycol dicaprylate/dicaprate | 6.50 | 4.50 | 3.00 |
| Butylene glycol | 4.50 | | 5.00 |
| Carbomer | | 0.30 | 0.35 |
| Dicaprylyl ether | 2.00 | | 1.50 |
| PVP/Hexadecene copolymer | 1.00 | | 0.50 |
| Xanthan gum | | 0.60 | 0.30 |
| Dimethicone | 1.00 | | |
| Cyclomethicone | | | |
| Tocopheryl acetate | | | 0.50 |
| Glycerol | | 3.50 | 2.00 |
| Dye (water- and/or oil soluble) | | | 0.15 |
| DMDM Hydantoin | | | 0.40 |
| Propylparaben | 0.25 | | |
| Methylparaben | 0.30 | | |
| Ethanol | | 1.50 | |
| Konkaben LMB ® | | 0.20 | |
| Trisodium EDTA | 1.00 | | 1.00 |
| Phenoxyethano1 | | 0.50 | 0.40 |
| Perfume | q.s. | q.s. | q.s. |
| Aqua | Ad 100 | Ad 100 | Ad 100 |

### EXAMPLES 7 TO 9

O/W Emulsions

| | Ex. 7 | Ex. 8 | Ex. 9 |
|---|---|---|---|
| Polyglyceryl 3-methylglucose distearate | 3.00 | | |
| Glyceryl stearate citrate | | | |
| Glyceryl stearate SE | | | 3.00 |
| PEG-40 Stearate | | | |
| PEG-100 Stearate | | | |
| Sorbitan stearate | 1.00 | | |
| Sodium cetearyl sulfate | | | 1.00 |
| Cetearyl polyglucoside | | 2.00 | |
| Caprylic/capric triglyceride | | | 1.50 |
| Cetearyl alcohol | | | |
| Cetyl alcohol | 1.00 | | |
| Stearyl alcohol | | 5.00 | |
| Behenyl alcohol | 1.00 | | |
| Octyldodecanol | | | |
| Mineral oil | | | |
| Compound I | 5.00 | 0.30 | 2.00 |
| Ethylhexyl methoxycinnamate | | 8.00 | |
| Bis-ethylhexyloxyphenol methoxyphenyl- | 2.00 | | 1.50 |
| triazine | | | |
| Butylmethoxydibenzoylmethane | | 3.00 | |
| Phenyldibenzimidazoltetrasulfonic acid | | | 1.50 |
| Ethylhexyltriazone | 2.50 | | |
| 4-Methylbenzylidene camphor | | 2.00 | |
| Octocrylene | | 5.00 | |
| Diethylhexylbutamidotriazone | | | 2.50 |
| Phenylbenzimidazolesulfonic acid | | | 2.00 |
| Methylenebis-benzotriazolyl tetramethyl- | 3.00 | | 2.50 |
| butylphenol | | | |
| Ethylhexyl salicylate | | | |
| Homosalate | 4.50 | | |
| Titanium dioxide silicone coating | | | 3.00 |
| Zinc oxide HP-1 | 2.00 | | |
| C₁₂—C₁₅-alkyl benzoate | 2.00 | | 1.50 |
| Dicaprylyl carbonate | | 3.00 | |
| Butylene glycol dicaprylate/dicaprate | 5.00 | | |
| Butylene glycol | | 7.50 | |
| Carbomer | 0.30 | 0.10 | 0.20 |
| Tocopheryl acetate | 0.30 | | 0.50 |
| Dicaprylyl ether | 2.50 | 0.50 | |
| PVP/Hexadecene copolymer | 1.00 | | 0.80 |
| Xanthan gum | 0.20 | | 0.40 |
| Dimethicone | 2.00 | | |
| Cyclomethicone | | 2.50 | |
| Glycerol | 5.00 | | 7.50 |
| Dye (water- and/or oil-soluble) | 0.15 | 0.25 | |
| DMDM Hydantoin | | | 0.20 |
| Parabens | 0.60 | | |
| Ethanol | | | |
| Konkaben LMB ® | | | |
| Trisodium EDTA | 1.00 | | 1.00 |
| Phenoxyethanol | 0.15 | | 0.30 |
| Perfume | q.s. | q.s. | q.s. |
| Aqua | Ad 100 | Ad 100 | Ad 100 |

### EXAMPLES 10 TO 13

O/W Emulsions

| | Ex. 10 | Ex. 11 | Ex. 12 | Ex. 13 |
|---|---|---|---|---|
| Polyglyceryl 3-methylglucose di- | | | | |
| stearate | | | | |
| Glyceryl stearate citrate | | | | 2.50 |
| Glyceryl stearate SE | 2.00 | | 3.00 | |
| PEG-40 Stearate | 1.00 | | | |
| PEG-100 Stearate | | | 1.00 | |
| Sorbitan stearate | | | | |
| Sodium cetearyl sulfate | | | | 0.50 |
| Cetearyl polyglucoside | | | | |
| Caprylic/capric triglyceride | | | | 1.00 |
| Cetearyl alcohol | 2.00 | | | |
| Cetyl alcohol | | | 1.00 | |
| Stearyl alcohol | | | | |
| Behenyl alcohol | | | | |
| Octyldodecanol | | | | 2.00 |
| Mineral oil | | | | 1.50 |
| Compound I | 6.00 | 0.50 | 5.00 | 0.20 |
| Ethylhexyl methoxycinnamate | | 3.00 | | 4.00 |
| Bis-ethylhexyloxyphenol methoxy- | | 2.00 | | 2.00 |
| phenyltriazine | | | | |
| Butylmethoxydibenzoylmethane | | | | 1.00 |
| Phenyldibenzimidazoltetrasulfonic | | | | 0.50 |
| acid | | | | |
| Ethylhexyltriazone | 4.00 | | | 2.00 |
| 4-Methylbenzylidene camphor | | | | |
| Octocrylene | | | | |
| Diethylhexylbutamidotriazone | | 1.50 | | 1.00 |
| Phenylbenzimidazolesulfonic acid | 1.00 | | | |
| Methylenebis-benzotriazolyl | 2.00 | | | |
| tetramethylbutylphenol | | | | |
| Ethylhexyl salicylate | | 4.00 | | |
| Homosalate | | | | |
| Titanium dioxide silicone coating | 2.00 | 1.00 | | 2.00 |
| Zinc oxide HP-1 | 3.00 | | | |
| C₁₂-C₁₅-alkyl benzoate | 2.50 | | | 0.80 |
| Dicaprylyl carbonate | | | 2.00 | |
| Butylene glycol dicaprylate/di- | 6.50 | | 4.50 | 3.00 |
| caprate | | | | |
| Butylene glycol | 4.50 | 9.00 | | 5.00 |
| Carbomer | | | 0.30 | 0.35 |
| Tocopheryl acetate | | | 0.50 | 0.80 |
| Dicaprylyl ether | 2.00 | | | 1.50 |
| PVP/Hexadecene copolymer | 1.00 | | | 0.50 |
| Xanthan gum | | 1.00 | 0.60 | 0.30 |
| Dimethicone | 1.00 | | | |
| Cyclomethicone | | 5.00 | | |
| Glycerol | | 4.00 | 3.50 | 2.00 |
| Dye (water- and/or oil-soluble) | 0.20 | | | 0.10 |
| DMDM Hydantoin | | | | 0.40 |
| Parabene | 0.30 | | | |
| Ethanol | | 5.00 | 1.50 | |
| Konkaben LMB ® | | | 0.20 | |
| Trisodium EDTA | 1.00 | 0.50 | | 1.00 |
| Phenoxyethanol | | | 0.50 | 0.40 |
| Perfume | q.s. | q.s. | q.s. | q.s. |
| Aqua | Ad 100 | Ad 100 | Ad 100 | Ad 100 |

We claim:

1. A cosmetic or dermatological preparation in the form of an O/W emulsion, comprising an amino-substituted hydroxybenzophenone of the formula I.

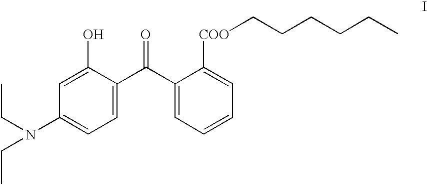

2. A preparation as claimed in claim 1, wherein the content of the amino-substituted hydroxybenzophenone of the formula I in the preparation is 0.01 to 20% by weight, based on the total weight of the preparation.

3. A preparation as claimed in either of claims 1 and 2, wherein the oil phase comprises lipids which have an interfacial tension toward water of less than 30 mN/m.

4. A preparation as claimed in claim 3, wherein the lipids of the oil phase are chosen from the group consisting of 2-ethylhexyl isostearate, octyldodecanol, isotridecyl isononanoate, isoeicosane, 2-ethylhexyl cocoate, C₁₂-C₁₅-alkyl benzoate, caprylic/capric triglyceride and dicaprylyl ether.

5. A preparation as claimed in claim 3, wherein the lipids of the oil phase are chosen from the group consisting of mixtures of octyldodecanol, caprylic/capric triglyceride and dicaprylyl ether or mixtures of C₁₂-C₁₅-alkyl benzoate and 2-ethylhexyl isostearate, mixtures of C₁₂-C₁₅-alkyl benzoate and isotridecyl isononanoate and mixtures of C₁₂-C₁₅-alkyl benzoate, 2-ethylhexyl isostearate and isotridecyl isononanoate.

6. A preparation as claimed in claim 3, wherein some or all of the lipids of the oil phase are chosen from cyclic and/or linear silicones.
